(19) United States
(12) Patent Application Publication
(10) Pub. No.: US 2021/0113511 A1
(43) Pub. Date: Apr. 22, 2021

(54) METHODS AND COMPOSITIONS FOR THE TREATMENT OF ACNE

(71) Applicant: Sol-Gel Technologies Ltd., Ness Ziona (IL)

(72) Inventors: Ofer Toledano, Kfar Saba (IL); Ofra Levy-Hacham, Ness Ziona (IL); Rinat Mizrahi, Bat-Yam (IL)

(73) Assignee: Sol-Gel Technologies Ltd., Ness Ziona (IL)

(21) Appl. No.: 17/137,883

(22) Filed: Dec. 30, 2020

Related U.S. Application Data
(63) Continuation-in-part of application No. 16/897,308, filed on Jun. 10, 2020.
Application No. 16/897,308 is a continuation of application No. 16/514,033, filed on Jul. 17, 2019, now Pat. No. 10,702,493.
Application No. 16/514,033 is a continuation of application No. 16/033,257, filed on Jul. 12, 2018, now Pat. No. 10,420,743.
(60) Provisional application No. 62/531,396, filed on Jul. 12, 2017.
(60) Provisional application No. 62/955,010, filed on Dec. 30, 2019.

(51) Int. Cl. A61K 31/203; A61K 9/00; A61P 17/10; A61K 9/50; A61K 31/327

(52) U.S. Cl. CPC A61K 31/203; A61K 9/006; A61K 31/327; A61K 9/0014; A61K 9/501; A61P 17/10

(57) ABSTRACT

The present application is directed to regimens, methods of treatment, and compositions for the treatment of acne in a subject suffering therefrom.

### CROSS REFERENCE TO RELATED APPLICATIONS

This application is a Continuation-in-Part Application of U.S. patent application Ser. No. 16/897,308 filed Jun. 10, 2020 which is a Continuation Application of U.S. patent application Ser. No. 16/514,033, filed Jul. 17, 2019, which is a Continuation Application of U.S. patent application Ser. No. 16/033,257, filed Jul. 12, 2018, which claims the benefit of U.S. Ser. No. 62/531,396, filed Jul. 12, 2017 and this invention claims the benefit from U.S. Ser. No. 62/955,010, filed Dec. 30, 2019 which are all incorporated in its entirety herein by reference.

### FIELD OF THE INVENTION

The present application is directed to regimens, methods of treatment and compositions for the treatment of acne in a subject suffering therefrom.

### BACKGROUND OF THE INVENTION

Acne vulgaris is a common condition of the pilo-sebaceous units of the skin (hair follicles and oil glands). Acne is the most common skin disorder in the United States, affecting 40-50 million Americans. Acne usually begins in puberty, but the condition is not restricted to any age group. Approximately 85% of people between the ages of 12 and 24 experience at least minor, most often on the face, chest, and back [Bhate and Williams].

Acne is caused by four major factors: (1) production of oil by enlarged oil glands in the skin, (2) blockage of the hair follicles that release oil, (3) growth of bacteria, called Propionibacterium acnes (P. acnes), within the hair follicles and (4) inflammatory/immune response to P. acnes.

The pathophysiologic features of acne suggest that combination therapy should be utilized as early as possible to simultaneously attack the multiple pathogenic factors of the condition [Gollnick and Cunliffe, J Am. Acad. Dermatol., 2003, 49 (1 Suppl):S1-37]. Antimicrobials have been a mainstay of acne treatment for many years, having multiple mechanisms of action. The most important may be the ability of antibiotics to decrease the number of P. acnes in and around the follicle. They have a bacteriostatic effect on P. acnes, which prevents the bacteria from producing pro-inflammatory molecules [Leyden et al. Semin. Cutaneous Med. Surg., 2001, 20(3): 139-143].

In clinical practice, it is common for physicians to prescribe multiple topical products for acne. Topical products are applied one or two times a day by the patient. However, many of these compounds are irritating with resultant development of facial erythema and discontinuation of the products or noncompliance with therapy. Benzoyl peroxide (BPO) and all trans retinoic acid (ATRA) are two active ingredients with different pharmacological actions that are commonly used for the treatment of acne.

Topical retinoids are keratinization inhibitors. They work by decreasing the cohesiveness of follicular epithelial cells. This, results in an inhibition in the formation of microcomedones, preventing the formation of mature comedones and inflammatory lesions [Gollnick and Cunliffe, J Am. Acad. Dermatol., 2003, 49 (1 Suppl):S1-37]. Use of retinoids promotes the normal desquamation of follicular epithelium. The action of the retinoid may enhance the penetration of other topical compounds used to treat acne.

BPO is a commonly used topical antibacterial agent for acne available either by prescription in combinations or over the counter (OTC). BPO has been found to be lethal to P. acnes as well as other bacteria that may reside on the skin. So far there has been no indication of any bacteria developing a resistance to BPO. It has also been demonstrated that BPO has keratolytic activity contributing to its efficacy in treating comedonal acne [Tanghetti, Cutis, 2008, 82 (5 Suppl):5-11]. BPO reduces the cohesiveness of the cells of the stratum corneum, thus improving topical drug delivery through the epidermal barrier.

A topically applied BPO acetone gel is clinically effective in the treatment of acne rosacea. Montes et al. (1983) Therapeutics Clin. 32:185-190. However, the rate of irritation reported by subjects administered BPO acetone gel was twice the rate for subjects administered a placebo gel. Gels were initially applied once daily, increasing to twice daily after abatement of initial irritation.

Silica microcapsule systems have been developed to overcome many of the limitations (such as degradation and irritation) of standard pharmaceutical formulations involving multiple active ingredients. The encapsulation of active ingredients in silica microcapsules serves to protect components in the formulation from interacting with one another and, as a consequence, increases overall formulation stability. Silica is chemically inert, photochemically and physically stable, and safe for topical use.

Applicant's silica encapsulated products meet the criteria for categorical exclusion defined in 21 CFR 25.31(e), and that to the knowledge of Applicant, no extraordinary circumstances exist as defined in 21 CFR 25.21. Thus, no environmental assessment is required according to 21 CFR 25.20(1). For the case of encapsulated BPO (E-BPO)/encapsulated ATRA (E-ATRA), microencapsulation of both BPO and tretinoin protects the tretinoin from oxidative decomposition by BPO, thereby enhancing the stability for this novel combination product and ensuring a suitable clinical and commercial shelf life (U.S. Pat. No. 8,617,580 and US 2012/0202695).

Clinicians have been reluctant to prescribe topical retinoids and BPO concurrently due to a belief that the BPO may result in oxidation and degradation of the tretinoin molecule, thereby reducing its effectiveness, and prefer to recommend the BPO or an antibiotic/BPO combination to be applied in the morning and tretinoin at night (Yan AC. Current concepts in acne management. Adolesc. Med. Clin. 2006; 17(3):613-637.)

Another publication (Emmy Graber, Treatment of Acne Vulgaris, UpToDate.com, July 2016) states “topical tretinoin should NOT be applied at the same time as benzoyl peroxide”, despite the known fact that newer retinoid compositions like Retin A microspheres (MICROSPONGE® System) have less interaction or no short-term interaction with BPO. Obviously, concomitant administration of tretinoin and BPO is taught away by this publication.

Unlike adapalene, which is often combined with BPO, tretinoin is significantly more irritant to the skin and since BPO is also irritant, it has been feared that the two APIs together will create unacceptable cutaneous side effects. Also, BPO is known to oxidize tretinoin and hence it was feared that their interaction on the skin when administered together will diminish the therapeutic effect of tretinoin. Thus, while there are some reports in the literature on the value of both compounds being administered one in the morning and the other in the evening, the verdict up to now was that the two products should not be administered concomitantly.

This belief of the medical profession explains why all previous attempts to solve the stability problem of tretinoin/BPO, such as microencapsulation technology, did not yield a commercial product so far.

Combination topical therapy is the recommended standard of care for the management of patients with acne [Gollnick and Cunliffe, J Am. Acad. Dermatol., 2003, 49 (1 Suppl):S1-37]. Combination therapy targets multiple pathogenic factors: abnormal follicular keratinization, P. acnes proliferation and inflammation. Combining the separate product applications into a single delivery system would provide the patient with the convenience of a single product, thus improving patient adherence and improving treatment outcomes.

### SUMMARY OF THE INVENTION

in some embodiments, this invention provides, a regimen for providing early onset of action in the treatment of acne comprising topically applying onto an affected skin area of a subject in need thereof, once a day for a period of time of at least about 4, 8 or 12 weeks, a topical medicament which comprises the active agents:
tretinoin or a pharmaceutically acceptable salt thereof, in an amount of about 0.01% to about 0.5% weight; and
benzoyl peroxide in an amount of between about 1% to about 10% weight
   and a pharmaceutically acceptable carrier or excipient, wherein the absolute reduction in non-inflammatory lesion count after about 2 weeks is at least about twice the absolute reduction in non-inflammatory lesion count during the period starting at about week 2 and ending at about week 4 of treatment.

In some embodiments, this invention provides a regimen for providing early onset of action in the treatment of acne comprising topically applying onto an affected skin area of a subject in need thereof, once a day for a period of time of at least about 2, 4, 8 or 12 weeks, a topical medicament which comprises the active agents:
tretinoin or a pharmaceutically acceptable salt thereof, in an amount of about 0.01% to about 0.5% weight; and
benzoyl peroxide in an amount of between about 1% to about 10% weight
   and a pharmaceutically acceptable carrier or excipient, to achieve a percentage decrease in the number of non-inflammatory lesions of about 25.2% (mean percentage change from baseline) after about 2 weeks, compared to a percentage decrease in the non-inflammatory lesions of about 17.8% after treatment with vehicle control for about 2 weeks.

In some embodiment, the regimen of the present invention provides an absolute reduction in non-inflammatory lesion count after about 2 weeks is at least about twice the absolute reduction in non-inflammatory lesion count during the period starting at about week 2 and ending at about week 4 of treatment. In some embodiment, the regimen of this invention provides, a reduction in total number of non-inflammatory lesion counts in a group of such subjects, wherein the reduction is a reduction of at least about 12 lesions after about 2 weeks of treatment. In some embodiment, the lesion are facial lesions.

In some embodiments, the regimen of the present invention provides a percentage decrease in the number of non-inflammatory lesions of about 25.2% (mean percentage change from baseline) after about 2 weeks, compared to a percentage decrease in the non-inflammatory lesions of about 17.8% after treatment with vehicle control for about 2 weeks.

In some embodiments, the regimen of the present invention provides a success rate after about 2 weeks that is at least twice the success rate after treatment with vehicle control for about 2 weeks, wherein the success rate is defined as at least a 2-grade improvement in Investigator Global Assessment (IGA) and clear or almost clear.

In some embodiments, in the regimen of the present invention, the score of at least one parameter evaluated by a Local Tolerability Score is synergistically lower than the score of the parameters evaluated with the same treatment regimen with each of the active agents separately. In another embodiment, the at least one parameter evaluated by the Local Tolerability score is selected from itching, burning, stinging, and any combinations thereof.

In some embodiments, the regimen provided herein, is directed to the treatment of acne, wherein the acne is mild acne, moderate acne, or severe acne.

In some embodiments, in the regimen of this invention, the at least one active agent of said medicament is encapsulated in a shell. In another embodiment, each of the active agents, BPO and tretinoin, of the medicament is encapsulated in its own, separate shell. In another embodiment, the shell is a metal oxide or semi-metal oxide inorganic shell.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 shows the HPLC chromatogram of an embodiment composition of the invention comprising 0.05% E-ATRA and 3% E-BPO eluted with acetonitrile and acetic acid 1% in water on a Zorbax RX-C18 3.5 mμ, 4.6*75 mm column, showing the RRT 0.44 product (all-trans 5,6-epoxy retinoic acid) at retention time of about 3.5 min (RRT product calculated relative to the ATRA peak at 7.8 min).

### DETAILED DESCRIPTION OF THE PRESENT INVENTION

When hypothesizing the development rationale of our tretinoin and benzoyl peroxide combination product, we expected that the efficacy of the combination at the end of the treatment to be superior over its monads-tretinoin alone and BPO alone. In the present invention, we have surprisingly found in the phase 3, where the combination was tested against vehicle, that the combination product (BPO and tretinoin) showed significantly earlier onset of action than the onset of action observed in the phase 2 for tretinoin alone or BPO alone. The early onset of action is reflected by the impressive reduction of both inflammatory and non-inflammatory lesion count. (In the case of IGA score, as this score relates to success rate, which in turn requires no lesions or almost no lesions, high IGA score is not expected in the middle of treatment, where the improvement is only partial).

Considering the chronic nature of acne, there is a need for early onset of action, and a prolonged use treatment of the disease, its symptoms and associated conditions, in a safe and effective manner. Thus, there exists a need for compositions that show early onset of action, and improved efficacy in the treatment of acne, that impart greater tolerance to the active principles and that reduce, substantially minimize or do not have the side effects described in the prior art.

In the first aspect, the present invention provides a regimen for the treatment of acne comprising: topically applying onto an affected skin area of a subject in need thereof, once a day for a period of time of up to 12 weeks, a topical medicament which comprises the active agents:
tretinoin or a pharmaceutically acceptable salt thereof, in an amount of between about 0.05% to about 0.1% weight; and
benzoyl peroxide in an amount of at least about 3% weight;

and a pharmaceutically acceptable carrier or excipient.

In some embodiments, the present invention provides a regimen for the treatment of acne, wherein the regimen comprises topically applying onto an affected skin area of a subject in need thereof, once a day for a period of at least about 4, 8 or 12 weeks, a topical medicament which comprises the active agents:
tretinoin or a pharmaceutically acceptable salt thereof, in an amount of between about 0.01% to about 0.5% weight; and
benzoyl peroxide in an amount of between about 1% w/w to about 10% weight;

and a pharmaceutically acceptable carrier or excipient.

In some embodiments, the present invention provides a regimen for early onset for the treatment of acne, wherein the regimen comprises topically applying onto an affected skin area of a subject in need thereof, once a day for a period of at least about 2, 4, 8 or 12 weeks, a topical medicament which comprises the active agents:
tretinoin or a pharmaceutically acceptable salt thereof, in an amount of between about 0.01% to about 0.5% weight; and
benzoyl peroxide in an amount of between about 1% w/w to about 10% weight;

and a pharmaceutically acceptable carrier or excipient.

In some embodiments, the present invention provides a regimen for early onset for the treatment of acne, wherein the regimen comprises topically applying onto an affected skin area of a subject in need thereof, once a day for a period of at least about 2, 4, 8 or 12 weeks, a topical medicament which comprises the active agents:
tretinoin or a pharmaceutically acceptable salt thereof, in an amount of between about 0.05% to about 0.1% weight; and
benzoyl peroxide in an amount of between about 1% w/w to about 10% weight;
and a pharmaceutically acceptable carrier or excipient.

In other embodiments, the regimen provides herein an absolute reduction in non-inflammatory lesion count after about 2 weeks is at least about twice the absolute reduction in non-inflammatory lesion count during the period starting at about week 2 and ending at about week 4 of treatment. In another embodiment, the regimen provides herein a reduction in the total number of non-inflammatory lesion counts of at least about 12 lesions after about 2 weeks of treatment. In another embodiments, the lesions are facial lesions.

In some embodiments, the regimen provides herein a success rate after about 4 weeks that is at least twice the success rate after treatment with vehicle control for about 4 weeks, wherein the success rate is defined as at least a 2-grade improvement in Investigator Global Assessment (IGA) and clear or almost clear.

In some embodiments, the success rate of the regimen provided herein is about 5.4% after treatment with the pharmaceutical composition for about 4 weeks, compared to a success rate of about 2.4% after treatment with vehicle control for about 4 weeks.

In some embodiments, the regimen provided herein decreases the percentage of the number of non-inflammatory lesions of about 25.2% (mean percentage change from baseline) after about 2 weeks, compared to a percentage decrease in the non-inflammatory lesions of about 17.8% after treatment with vehicle control for about 2 weeks. In another embodiment, a reduction in the total number of non-inflammatory lesion counts in a group of such subjects is a reduction of at least about 12 lesions after about 2 weeks of treatment.

In some embodiments, the regimen provided herein, decreases the percentage of inflammatory lesions of about 27.1% (mean percentage change from of number baseline) after about 2 weeks, compared to a percentage decrease in the inflammatory lesions of about 19.5% after treatment with vehicle control for about 2 weeks. In another embodiment, a reduction in total number of inflammatory lesions counts in a group of such subjects is a reduction of at least about 8 lesions after about 2 weeks of treatment.

In some embodiments, the regimen provided herein, provides a success rate of at least about 2.3% after about 2 weeks, at least about 8.7% after about 4 weeks, at least about 21.6% after about 8 weeks, or at least about 43.2% after about 12 weeks, wherein the success rate is defined as at least a 2-grade improvement in Investigator Global Assessment (IGA).

In some embodiments, the regimen provided herein, provides a success rate after about 2 weeks that is at least twice the success rate after treatment with vehicle control for about 2 weeks, wherein the success rate is defined as at least a 2-grade improvement in Investigator Global Assessment (IGA) and clear or almost clear.

In some embodiments, the pharmaceutical composition provided herein is applied once daily for a period of about 2 weeks. In some embodiments, the pharmaceutical composition provided herein is applied once daily for a period of about 4 weeks. In some embodiments, the pharmaceutical composition provided herein is applied once daily for a period of about 8 weeks. In some embodiments, the pharmaceutical composition provided herein is applied once daily for a period of at least 12 weeks.

In some embodiments, this invention provides a regimen for providing early onset of action in the treatment of acne comprising topically applying onto an affected skin area of a subject in need thereof, once a day for a period of time of at least about 4, 8 or 12 weeks, a topical medicament which comprises the active agents:
tretinoin or a pharmaceutically acceptable salt thereof, in an amount of about 0.01% to about 0.5% weight; and
benzoyl peroxide in an amount of between about 1% to about 10% weight
   and a pharmaceutically acceptable carrier or excipient, wherein the absolute reduction in non-inflammatory lesion count after about 2 weeks is at least about twice the absolute reduction in non-inflammatory lesion count during the period starting at about week 2 and ending at about week 4 of treatment. In another embodiments, the lesions are facial lesions. In another embodiment, the topical medicament is applied once daily for a period of about 4 weeks. In another embodiments, the topical medicament is applied once daily for a period of about 8 weeks. In another embodiments, the topical medicament is applied once daily for a period of about 12 weeks. In another embodiment, the benzoyl peroxide and the tretinoin or a pharmaceutically acceptable salt thereof are the sole active agent administered to the subject in need of said treatment during the duration of the regimen. In another embodiment, the topical medicament is applied to the face. In another embodiment, the acne is any of mild acne, moderate acne, or severe acne.

In some embodiments, this invention provides a regimen for providing early onset of action in the treatment of acne comprising topically applying onto an affected skin area of a subject in need thereof, once a day for a period of time of at least about 4, 8 or 12 weeks, a topical medicament which comprises the active agents:
tretinoin or a pharmaceutically acceptable salt thereof, in an amount of about 0.01% to about 0.5% weight; and
benzoyl peroxide in an amount of between about 1% to about 10% weight
   and a pharmaceutically acceptable carrier or excipient, wherein the absolute reduction in non-inflammatory lesion count after about 2 weeks is at least about twice the absolute reduction in non-inflammatory lesion count during the period starting at about week 2 and ending at about week 4 of treatment; wherein the reduction in the total number of non-inflammatory lesion counts is a reduction of at least about 12 lesions after about 2 weeks of treatment. In another embodiment, the topical medicament is applied once daily for a period of about 4 weeks. In another embodiments, the topical medicament is applied once daily for a period of about 8 weeks. In another embodiments, the topical medicament is applied once daily for a period of about 12 weeks. In another embodiment, the benzoyl peroxide and the tretinoin or a pharmaceutically acceptable salt thereof are the sole active agent administered to the subject in need of said treatment during the duration of the regimen. In another embodiment, the topical medicament is applied to the face. In another embodiment, the acne is any of mild acne, moderate acne, or severe acne.

In some embodiments, this invention provides a regimen for providing early onset of action in the treatment of acne comprising topically applying onto an affected skin area of a subject in need thereof, once a day for a period of time of at least about 4, 8 or 12 weeks, a topical medicament which comprises the active agents:
tretinoin or a pharmaceutically acceptable salt thereof, in an amount of about 0.01% to about 0.5% weight; and
benzoyl peroxide in an amount of between about 1% to about 10% weight
   and a pharmaceutically acceptable carrier or excipient, wherein the absolute reduction in non-inflammatory lesion count after about 2 weeks is at least about twice the absolute reduction in non-inflammatory lesion count during the period starting at about week 2 and ending at about week 4 of treatment; wherein the regimen provides a success rate after about 4 weeks that is at least twice the success rate after treatment with vehicle control for about 4 weeks, wherein the success rate is defined as at least a 2-grade improvement in Investigator Global Assessment (IGA) and clear or almost clear. In another embodiment, the topical medicament is applied once daily for a period of about 4 weeks. In another embodiments, the topical medicament is applied once daily for a period of about 8 weeks. In another embodiments, the topical medicament is applied once daily for a period of about 12 weeks. In another embodiment, the benzoyl peroxide and the tretinoin or a pharmaceutically acceptable salt thereof are the sole active agent administered to the subject in need of said treatment during the duration of the regimen. In another embodiment, the topical medicament is applied to the face. In another embodiment, the acne is any of mild acne, moderate acne, or severe acne. In another embodiment, the success rate of the regimen provided herein is about 5.4% after treatment with the pharmaceutical composition for about 4 weeks, compared to a success rate of about 2.4% after treatment with vehicle control for about 4 weeks.

In some embodiments, this invention provides a regimen for providing early onset of action in the treatment of acne comprising topically applying onto an affected skin area of a subject in need thereof, once a day for a period of time of at least about 2, 4, 8 or 12 weeks, a topical medicament which comprises the active agents:
tretinoin or a pharmaceutically acceptable salt thereof, in an amount of about 0.01% to about 0.5% weight; and
benzoyl peroxide in an amount of between about 1% to about 10% weight
   and a pharmaceutically acceptable carrier or excipient, to achieve a percentage decrease in the number of non-inflammatory lesions of about 25.2% (mean percentage change from baseline) after about 2 weeks, compared to a percentage decrease in the non-inflammatory lesions of about 17.8% after treatment with vehicle control for about 2 weeks. In another embodiments, the lesions are facial lesions. In another embodiment, the topical medicament is applied once daily for a period of about 2 weeks. In another embodiment, the topical medicament is applied once daily for a period of about 4 weeks. In another embodiments, the topical medicament is applied once daily for a period of about 8 weeks. In another embodiments, the topical medicament is applied once daily for a period of about 12 weeks. In another embodiment, the benzoyl peroxide and the tretinoin or a pharmaceutically acceptable salt thereof are the sole active agent administered to the subject in need of said treatment during the duration of the regimen. In another embodiment, the topical medicament is applied to the face. In another embodiment, the acne is any of mild acne, moderate acne, or severe acne.

In some embodiments, this invention provides a regimen for providing early onset of action in the treatment of acne comprising topically applying onto an affected skin area of a subject in need thereof, once a day for a period of time of at least about 2, 4, 8 or 12 weeks, a topical medicament which comprises the active agents:
tretinoin or a pharmaceutically acceptable salt thereof, in an amount of about 0.01% to about 0.5% weight; and
benzoyl peroxide in an amount of between about 1% to about 10% weight
   and a pharmaceutically acceptable carrier or excipient, to achieve a percentage decrease in the number of non-inflammatory lesions of about 25.2% (mean percentage change from baseline) after about 2 weeks, compared to a percentage decrease in the non-inflammatory lesions of about 17.8% after treatment with vehicle control for about 2 weeks, wherein the reduction in the total number of non-inflammatory lesion counts is a reduction of at least about 12 lesions after about 2 weeks of treatment. In another embodiments, the lesions are facial lesions. In another embodiment, the topical medicament is applied once daily for a period of about 2 weeks. In another embodiment, the topical medicament is applied once daily for a period of about 4 weeks. In another embodiments, the topical medicament is applied once daily for a period of about 8 weeks. In another embodiments, the topical medicament is applied once daily for a period of about 12 weeks. In another embodiment, the benzoyl peroxide and the tretinoin or a pharmaceutically acceptable salt thereof are the sole active agent administered to the subject in need of said treatment during the duration of the regimen. In another embodiment, the topical medicament is applied to the face. In another embodiment, the acne is any of mild acne, moderate acne, or severe acne.

In some embodiments, this invention provides a regimen for providing early onset of action in the treatment of acne comprising topically applying onto an affected skin area of a subject in need thereof, once a day for a period of time of at least about 2, 4, 8 or 12 weeks, a topical medicament which comprises the active agents:
tretinoin or a pharmaceutically acceptable salt thereof, in an amount of about 0.01% to about 0.5% weight; and
benzoyl peroxide in an amount of between about 1% to about 10% weight

and a pharmaceutically acceptable carrier or excipient, to achieve a percentage decrease in the number of non-inflammatory lesions of about 25.2% (mean percentage change from baseline) after about 2 weeks, compared to a percentage decrease in the non-inflammatory lesions of about 17.8% after treatment with vehicle control for about 2 weeks, wherein the percentage decrease of the inflammatory lesions is of about 27.1% (mean percentage change from of number baseline) after about 2 weeks, compared to a percentage decrease in the inflammatory lesions of about 19.5% after treatment with vehicle control for about 2 weeks. In another embodiment, a reduction in total number of inflammatory lesions counts in a group of such subjects is a reduction of at least about 8 lesions after about 2 weeks of treatment. In another embodiments, the lesions are facial lesions. In another embodiment, the topical medicament is applied once daily for a period of about 2 weeks. In another embodiment, the topical medicament is applied once daily for a period of about 4 weeks. In another embodiments, the topical medicament is applied once daily for a period of about 8 weeks. In another embodiments, the topical medicament is applied once daily for a period of about 12 weeks. In another embodiment, the benzoyl peroxide and the tretinoin or a pharmaceutically acceptable salt thereof are the sole active agent administered to the subject in need of said treatment during the duration of the regimen. In another embodiment, the topical medicament is applied to the face. In another embodiment, the acne is any of mild acne, moderate acne, or severe acne.

In some embodiments, this invention provides a regimen for providing early onset of action in the treatment of acne comprising topically applying onto an affected skin area of a subject in need thereof, once a day for a period of time of at least about 2, 4, 8 or 12 weeks, a topical medicament which comprises the active agents:
tretinoin or a pharmaceutically acceptable salt thereof, in an amount of about 0.01% to about 0.5% weight; and
benzoyl peroxide in an amount of between about 1% to about 10% weight
   and a pharmaceutically acceptable carrier or excipient, to achieve a percentage decrease in the number of non-inflammatory lesions of about 25.2% (mean percentage change from baseline) after about 2 weeks, compared to a percentage decrease in the non-inflammatory lesions of about 17.8% after treatment with vehicle control for about 2 weeks, wherein, the regimen provides a success rate of at least about 2.3% after about 2 weeks, at least about 8.7% after about 4 weeks, at least about 21.6% after about 8 weeks, or at least about 43.2% after about 12 weeks, wherein the success rate is defined as at least a 2-grade improvement in Investigator Global Assessment (IGA). In another embodiment, the regimen provided herein, provides a success rate after about 2 weeks that is at least twice the success rate after treatment with vehicle control for about 2 weeks, wherein the success rate is defined as at least a 2-grade improvement in Investigator Global Assessment (IGA) and clear or almost clear. In another embodiments, the lesions are facial lesions. In another embodiment, the topical medicament is applied once daily for a period of about 2 weeks. In another embodiment, the topical medicament is applied once daily for a period of about 4 weeks. In another embodiments, the topical medicament is applied once daily for a period of about 8 weeks. In another embodiments, the topical medicament is applied once daily for a period of about 12 weeks. In another embodiment, the benzoyl peroxide and the tretinoin or a pharmaceutically acceptable salt thereof are the sole active agent administered to the subject in need of said treatment during the duration of the regimen. In another embodiment, the topical medicament is applied to the face. In another embodiment, the acne is any of mild acne, moderate acne, or severe acne.

The term “topical medicament” and “pharmaceutical composition” are used herein interchangeably.

In a further aspect, the present invention provides a regimen for the treatment of acne comprising topically applying onto an affected skin area of a subject in need thereof, once a day for a period of time of up to 12 weeks, a topical medicament which comprises the active agents:
tretinoin or a pharmaceutically acceptable salt thereof, in an amount of between about 0.05% to about 0.1% weight; and
benzoyl peroxide in an amount of at least about 3% weight;

wherein the score of at least one parameter evaluated by an Investigator Cutaneous Safety Assessment is synergistically lower than the score of the parameters evaluated with the same treatment regimen with each of the active agents separately. In another embodiment, the topical medicament comprises about 0.1% tretinoin and about 3% benzoyl peroxide.

In some embodiments, the present invention provides a regimen for early onset for the treatment of acne, wherein the regimen comprises topically applying onto an affected skin area of a subject in need thereof, once a day for a period of time of at least about 2, 4, 8, or 12 weeks, a topical medicament which comprises the active agents:
tretinoin or a pharmaceutically acceptable salt thereof, in an amount of between about 0.05% to about 0.1% weight; and
benzoyl peroxide in an amount of at least about 3% weight; and a pharmaceutically acceptable carrier or excipient, and

wherein the score of at least one parameter evaluated by an Investigator Cutaneous Safety Assessment is synergistically lower than the score of the parameters evaluated with the same treatment regimen with each of the active agents separately. In another embodiment, the topical medicament comprises about 0.1% tretinoin and about 3% benzoyl peroxide.

In some embodiments, the present invention provides a regimen for early onset for the treatment of acne, wherein the regimen comprises topically applying onto an affected skin area of a subject in need thereof, once a day for a period of time of at least about 2, 4, 8, or 12 weeks, a topical medicament which comprises the active agents:
tretinoin or a pharmaceutically acceptable salt thereof, in an amount of between about 0.01% to about 0.5% weight; and
benzoyl peroxide in an amount of between about 1% to about 10% weight; and a pharmaceutically acceptable carrier or excipient, and

wherein the score of at least one parameter evaluated by an Investigator Cutaneous Safety Assessment is synergistically lower than the score of the parameters evaluated with the same treatment regimen with each of the active agents separately.

In some embodiments, this invention provides a regimen for providing early onset of action in the treatment of acne comprising topically applying onto an affected skin area of a subject in need thereof, once a day for a period of time of at least about 4, 8 or 12 weeks, a topical medicament which comprises the active agents:
tretinoin or a pharmaceutically acceptable salt thereof, in an amount of about 0.01% to about 0.5% weight; and
benzoyl peroxide in an amount of between about 1% to about 10% weight
   and a pharmaceutically acceptable carrier or excipient, wherein the absolute reduction in non-inflammatory lesion count after about 2 weeks is at least about twice the absolute reduction in non-inflammatory lesion count during the period starting at about week 2 and ending at about week 4 of treatment; wherein the score of at least one parameter evaluated by an Investigator Cutaneous Safety Assessment is synergistically lower than the score of the parameters evaluated with the same treatment regimen with each of the active agents separately. In another embodiment, the at least one parameter evaluated by the Investigator Cutaneous Safety Assessment is selected from erythema, dryness, scaling, pigmentation and any combination thereof. In another embodiments, the lesions are facial lesions. In another embodiment, the topical medicament is applied once daily for a period of about 4 weeks. In another embodiments, the topical medicament is applied once daily for a period of about 8 weeks. In another embodiments, the topical medicament is applied once daily for a period of about 12 weeks. In another embodiment, the benzoyl peroxide and the tretinoin or a pharmaceutically acceptable salt thereof are the sole active agent administered to the subject in need of said treatment during the duration of the regimen. In another embodiment, the topical medicament is applied to the face. In another embodiment, the acne is any of mild acne, moderate acne, or severe acne.

In some embodiments, this invention provides a regimen for providing early onset of action in the treatment of acne comprising topically applying onto an affected skin area of a subject in need thereof, once a day for a period of time of at least about 2, 4, 8 or 12 weeks, a topical medicament which comprises the active agents:
tretinoin or a pharmaceutically acceptable salt thereof, in an amount of about 0.01% to about 0.5% weight; and
benzoyl peroxide in an amount of between about 1% to about 10% weight
   and a pharmaceutically acceptable carrier or excipient, to achieve a percentage decrease in the number of non-inflammatory lesions of about 25.2% (mean percentage change from baseline) after about 2 weeks, compared to a percentage decrease in the non-inflammatory lesions of about 17.8% after treatment with vehicle control for about 2 weeks; wherein the score of at least one parameter evaluated by an Investigator Cutaneous Safety Assessment is synergistically lower than the score of the parameters evaluated with the same treatment regimen with each of the active agents separately. In another embodiment, the at least one parameter evaluated by the Investigator Cutaneous Safety Assessment is selected from erythema, dryness, scaling, pigmentation and any combination thereof. In another embodiments, the lesions are facial lesions. In another embodiment, the topical medicament is applied once daily for a period of about 2 weeks. In another embodiment, the topical medicament is applied once daily for a period of about 4 weeks. In another embodiments, the topical medicament is applied once daily for a period of about 8 weeks. In another embodiments, the topical medicament is applied once daily for a period of about 12 weeks. In another embodiment, the benzoyl peroxide and the tretinoin or a pharmaceutically acceptable salt thereof are the sole active agent administered to the subject in need of said treatment during the duration of the regimen. In another embodiment, the topical medicament is applied to the face. In another embodiment, the acne is any of mild acne, moderate acne, or severe acne.

In some embodiments, the at least one parameter evaluated by the Investigator Cutaneous Safety Assessment is selected from Erythema, Scaling, Pigmentation and any combinations thereof.

In yet another aspect, the present invention provides a regimen for the treatment of acne comprising topically applying onto an affected skin area of a subject in need thereof, once a day for a period of time of up to 12 weeks, a topical medicament which comprises the active agents:
tretinoin or a pharmaceutically acceptable salt thereof, in an amount of between about 0.05% to about 0.1% weight; and
benzoyl peroxide in an amount of at least about 3% weight;

wherein the score of at least one parameter evaluated by a Local Tolerability Score is synergistically lower than the score of the parameters evaluated with the same treatment regimen with each of the active agents separately. In another embodiment, the topical medicament comprises about 0.1% tretinoin and about 3% benzoyl peroxide.

In some embodiments, the present invention provides a regimen for the treatment of acne comprising topically applying onto an affected skin area of a subject in need thereof, once a day for a period of time of at least about 2, 4, 8 or 12 weeks, a topical medicament which comprises the active agents:
tretinoin or a pharmaceutically acceptable salt thereof, in an amount of between about 0.05% to about 0.1% weight; and
benzoyl peroxide in an amount of at least about 3% weight; and a pharmaceutically acceptable carrier or excipient; and

wherein the score of at least one parameter evaluated by a Local Tolerability Score is synergistically lower than the score of the parameters evaluated with the same treatment regimen with each of the active agents separately. In another embodiment, the topical medicament comprises about 0.1% tretinoin and about 3% benzoyl peroxide.

In some embodiments, the score of at least one parameter evaluated by a Local Tolerability Score can be similar to or lower than the score of the parameter evaluated with the same treatment regimen with a vehicle control.

In some embodiments, the present invention provides a regimen a regimen for early onset for the treatment of acne, wherein the regimen comprises topically applying onto an affected skin area of a subject in need thereof, once a day for a period of time of at least about 2, 4, 8 or 12 weeks, a topical medicament which comprises the active agents:
tretinoin or a pharmaceutically acceptable salt thereof, in an amount of between about 0.01% to about 0.5% weight; and
benzoyl peroxide in an amount of between about 1% to about 10% weight; and a pharmaceutically acceptable carrier or excipient; and

wherein the score of at least one parameter evaluated by a Local Tolerability Score is synergistically lower than the score of the parameters evaluated with the same treatment regimen with each of the active agents separately. In another embodiment, the pharmaceutical composition also comprises a pharmaceutically acceptable carrier or excipient.

In another embodiment, the score of at least one parameter evaluated by a Local Tolerability Score can be similar to or lower than the score of the parameter evaluated with the same treatment regimen with a vehicle control.

In some embodiments, the adverse events value, the parameter evaluated by an Investigator Cutaneous Safety Assessment, or the parameter evaluated by a Local Tolerability Score can be assessed at period of about 2, 4, 8 or 12 weeks.

In some embodiments, the regimen has an adverse events value similar to or lower than the adverse events value of a vehicle control.

In some embodiments, the score of at least one parameter evaluated by an Investigator Cutaneous Safety Assessment can be similar to or lower than the score of the parameters evaluated with the same treatment regimen with a vehicle control. For example, the at least one parameter evaluated by the Investigator Cutaneous Safety Assessment can be selected from erythema, dryness, scaling, pigmentation and any combinations thereof.

In some embodiments, this invention provides a regimen for providing early onset of action in the treatment of acne comprising topically applying onto an affected skin area of a subject in need thereof, once a day for a period of time of at least about 4, 8 or 12 weeks, a topical medicament which comprises the active agents:
tretinoin or a pharmaceutically acceptable salt thereof, in an amount of about 0.01% to about 0.5% weight; and
benzoyl peroxide in an amount of between about 1% to about 10% weight
   and a pharmaceutically acceptable carrier or excipient, wherein the absolute reduction in non-inflammatory lesion count after about 2 weeks is at least about twice the absolute reduction in non-inflammatory lesion count during the period starting at about week 2 and ending at about week 4 of treatment; wherein the score of at least one parameter evaluated by an Local Tolerability Score is similar to or lower than the score of the parameter evaluated with the same treatment regimen with a vehicle control. In another embodiment, the at least one parameter evaluated by the Local Tolerability Score is selected from itching, burning, stinging, and any combination thereof. In another embodiments, the lesions are facial lesions. In another embodiment, the topical medicament is applied once daily for a period of about 4 weeks. In another embodiments, the topical medicament is applied once daily for a period of about 8 weeks. In another embodiments, the topical medicament is applied once daily for a period of about 12 weeks. In another embodiment, the benzoyl peroxide and the tretinoin or a pharmaceutically acceptable salt thereof are the sole active agent administered to the subject in need of said treatment during the duration of the regimen. In another embodiment, the topical medicament is applied to the face. In another embodiment, the acne is any of mild acne, moderate acne, or severe acne.

In some embodiments, this invention provides a regimen for providing early onset of action in the treatment of acne comprising topically applying onto an affected skin area of a subject in need thereof, once a day for a period of time of at least about 2, 4, 8 or 12 weeks, a topical medicament which comprises the active agents:
tretinoin or a pharmaceutically acceptable salt thereof, in an amount of about 0.01% to about 0.5% weight; and
benzoyl peroxide in an amount of between about 1% to about 10% weight
   and a pharmaceutically acceptable carrier or excipient, to achieve a percentage decrease in the number of non-inflammatory lesions of about 25.2% (mean percentage change from baseline) after about 2 weeks, compared to a percentage decrease in the non-inflammatory lesions of about 17.8% after treatment with vehicle control for about 2 weeks; wherein the score of at least one parameter evaluated by an Local Tolerability Score is similar to or lower than the score of the parameter evaluated with the same treatment regimen with a vehicle control. In another embodiment, the at least one parameter evaluated by the Local Tolerability Score is selected from itching, burning, stinging, and any combination thereof. In another embodiments, the lesions are facial lesions. In another embodiment, the topical medicament is applied once daily for a period of about 2 weeks. In another embodiment, the topical medicament is applied once daily for a period of about 4 weeks. In another embodiments, the topical medicament is applied once daily for a period of about 8 weeks. In another embodiments, the topical medicament is applied once daily for a period of about 12 weeks. In another embodiment, the benzoyl peroxide and the tretinoin or a pharmaceutically acceptable salt thereof are the sole active agent administered to the subject in need of said treatment during the duration of the regimen. In another embodiment, the topical medicament is applied to the face. In another embodiment, the acne is any of mild acne, moderate acne, or severe acne.

In some embodiments, the at least one parameter evaluated by the Local Tolerability score is selected from itching, burning, stinging, and any combinations thereof.

In a further aspect, the present invention provides a regimen for the treatment of acne comprising topically applying onto an affected skin area of a subject in need thereof, once a day for a period of time of up to 12 weeks, a topical medicament which comprises the active agents:
tretinoin or a pharmaceutically acceptable salt thereof, in an amount of between about 0.05% to about 0.1% weight; and
benzoyl peroxide in an amount of at least about 3% weight;

wherein said regimen improves the IGA success rate by at least 20% compared to the baseline score. In another embodiment, the topical medicament comprises about 0.1% tretinoin and about 3% benzoyl peroxide.

In some aspects, the present invention provides a regimen for the treatment of acne comprising topically applying onto an affected skin area of a subject in need thereof, once a day for a period of time of at least 2, 4, 8, or 12 weeks, a topical medicament which comprises the active agents:
tretinoin or a pharmaceutically acceptable salt thereof, in an amount of between about 0.05% to about 0.1% weight; and
benzoyl peroxide in an amount of at least about 3% weight; and a pharmaceutically acceptable carrier or excipient; and

wherein said regimen improves the IGA success rate by at least 20% compared to the baseline score.

In another aspect, the present invention provides a regimen for the treatment of acne comprising topically applying onto an affected skin area of a subject in need thereof, once a day for a period of time of at least 2, 4, 8, or 12 weeks, a topical medicament which comprises the active agents:
tretinoin or a pharmaceutically acceptable salt thereof, in an amount of between about 0.01% to about 0.5% weight; and
benzoyl peroxide in an amount of between about 1% to about 10% weight;

wherein said regimen improves the IGA success rate by at least 20% compared to the baseline score.

In some embodiments, the present invention provides a regimen for the treatment of acne comprising topically applying onto an affected skin area of a subject in need thereof, once a day for a period of time of at least 2, 4, 8, or 12 weeks, a topical medicament which comprises the active agents:
tretinoin or a pharmaceutically acceptable salt thereof, in an amount of between about 0.01% to about 0.5% weight; and
benzoyl peroxide in an amount of between about 1% to about 10% weight;

and a pharmaceutically acceptable carrier or excipient, wherein the regimen provides, a success rate after about 4 weeks that is at least twice the success rate after treatment with vehicle control for about 4 weeks, wherein the success rate is defined as at least a 2-grade improvement in Investigator Global Assessment (IGA) and clear or almost clear.

In some embodiments, the success rate is about 5.4% after treatment with the pharmaceutical composition for about 4 weeks, compared to a success rate of about 2.4% after treatment with vehicle control for about 4 weeks.

In some embodiments, the present invention provides a regimen for the treatment of acne comprising topically applying onto an affected skin area of a subject in need thereof, once a day for a period of time of at least 2, 4, 8, or 12 weeks, a topical medicament which comprises the active agents:
tretinoin or a pharmaceutically acceptable salt thereof, in an amount of between about 0.01% to about 0.5% weight; and
benzoyl peroxide in an amount of between about 1% to about 10% weight;

and a pharmaceutically acceptable carrier or excipient, wherein the regimen provides, a percentage decrease in the number of non-inflammatory lesions of about 25.2% (mean percentage change from baseline) after about 2 weeks, compared to a percentage decrease in the non-inflamatory lesions of about 17.8% after treatment with vehicle control for about 2 weeks.

In some embodiments, a reduction in total number of non-inflammatory lesion counts in a group of such subjects is a reduction of at least about 12 lesions after about 2 weeks of treatment.

In some embodiments, the present invention provides a regimen for early onset for the treatment of acne, wherein the regimen comprises topically applying onto an affected skin area of a subject in need thereof, once a day for a period of time of at least 2, 4, 8, or 12 weeks, a topical medicament which comprises the active agents:
tretinoin or a pharmaceutically acceptable salt thereof, in an amount of between about 0.01% to about 0.5% weight; and
benzoyl peroxide in an amount of between about 1% to about 10% weight;

and a pharmaceutically acceptable carrier or excipient, wherein the regimen provides, a percentage decrease in the number of inflammatory lesions of about 27.1% (mean percentage change from baseline) after about 2 weeks, compared to a percentage decrease in the inflammatory lesions of about 19.5% after treatment with vehicle control for about 2 weeks,

In some embodiments, a reduction in total number of inflammatory lesions counts in a group of such subjects is a reduction of at least about 8 lesions after about 2 weeks of treatment. In some embodiments, the lesions can be facial lesions.

In some embodiments, the present invention provides a regimen for early onset for the treatment of acne, wherein the regimen comprises topically applying onto an affected skin area of a subject in need thereof, once a day for a period of time of at least 2, 4, 8, or 12 weeks, a topical medicament which comprises the active agents:
tretinoin or a pharmaceutically acceptable salt thereof, in an amount of between about 0.01% to about 0.5% weight; and
benzoyl peroxide in an amount of between about 1% to about 10% weight;

and a pharmaceutically acceptable carrier or excipient, wherein the regimen provides, a success rate of at least about 2.3% after about 2 weeks, at least about 8.7% after about 4 weeks, at least about 21.6% after about 8 weeks, or at least about 43.2% after about 12 weeks, wherein the success rate is defined as at least a 2-grade improvement in Investigator Global Assessment (IGA).

In some embodiments, the present invention provides a regimen for early onset for the treatment of acne, wherein the regimen comprises topically applying onto an affected skin area of a subject in need thereof, once a day for a period of time of at least 2, 4, 8, or 12 weeks, a topical medicament which comprises the active agents:
tretinoin or a pharmaceutically acceptable salt thereof, in an amount of between about 0.01% to about 0.5% weight; and
benzoyl peroxide in an amount of between about 1% to about 10% weight;

and a pharmaceutically acceptable carrier or excipient, wherein the regimen provides, a success rate after about 2 weeks that is at least twice the success rate after treatment with vehicle control for about 2 weeks, wherein the success rate is defined as at least a 2-grade improvement in Investigator Global Assessment (IGA) and clear or almost clear.

In some embodiments, the present invention provides a regimen for early onset for the treatment of acne, wherein the regimen comprises topically applying onto an affected skin area of a subject in need thereof, once a day for a period of time of at least 2, 4, 8, or 12 weeks, a topical medicament which comprises the active agents:
tretinoin or a pharmaceutically acceptable salt thereof, in an amount of between about 0.01% to about 0.5% weight; and
benzoyl peroxide in an amount of between about 1% to about 10% weight;

and a pharmaceutically acceptable carrier or excipient, wherein the subject has severe acne, wherein the regimen provides, in a group of such subjects, a success rate of at least about 50% after about 2 weeks, wherein the success rate is defined as at least a I-grade improvement in Investigator Global Assessment (IGA).

In some embodiments, the subject is 9 years of age or older. In some embodiments, the subject is less than 18 years of age. For example, the subject can be 9-11 years of age or 12-17 years of age, each inclusive. In some exemplary embodiments, the subject can be 18 years of age or older. In some embodiments, the subject can be 18-30 years of age, inclusive, or 31 years of an or older.

In some embodiments, the present invention provides a regimen for the treatment of acne comprising topically applying onto an affected skin area of a subject in need thereof, once a day for a period of time of at least 2, 4, 8, or 12 weeks, a topical medicament which comprises the active agents:
tretinoin or a pharmaceutically acceptable salt thereof, in an amount of between about 0.01% to about 0.5% weight; and
benzoyl peroxide in an amount of between about 1% to about 10% weight;

and a pharmaceutically acceptable carrier or excipient, wherein the subject is 9-11 years of age, wherein the regimen provides, in a group of such subjects, a success rate of at least about 60% after about 12 weeks, wherein the success rate is defined as at least a 2-grade improvement in Investigator Global Assessment (WA) and clear or almost clear.

In some embodiments, the present invention provides a regimen for early onset for the treatment of acne, wherein the regimen comprises topically applying onto an affected skin area of a subject in need thereof, once a day for a period of time of at least 2, 4, 8, or 12 weeks, a topical medicament which comprises the active agents:
tretinoin or a pharmaceutically acceptable salt thereof, in an amount of between about 0.01% to about 0.5% weight; and
benzoyl peroxide in an amount of between about 1% to about 10% weight;

and a pharmaceutically acceptable carrier or excipient, wherein the subject is 9-1.1 years of age, wherein the regimen provides, in a group of such subjects, a success rate of at least about 60% after about 12 weeks, wherein the success rate is defined as at least a 2-grade improvement in Investigator Global Assessment (IGA) and clear or almost clear.

In some embodiments, said regimen improves the IGA success rate by reducing the number of acne lesions and improving the clinical condition of a patient in need thereof as compared to their baseline condition/score.

In another one of its aspects, the present invention provides a regimen for the treatment of acne, comprising topically applying onto an affected skin area of a subject in need thereof, once a day for a period of time of up to 12 weeks, a topical medicament which comprises tretinoin or a pharmaceutically acceptable salt thereof, as a single active agent in an amount of between about 0.05% to about 0.1% weight; wherein said active agent is encapsulated in a shell; and

wherein the score of at least one parameter evaluated by an Investigator Cutaneous Safety Assessment is lower than the score of the parameters evaluated with the same treatment regimen using a non-encapsulated active agent. In another embodiment, the topical medicament comprises about 0.1% w/w of tretinoin and about 3% w/w of benzoyl peroxide.

In another one of its aspects, the present invention provides a regimen for the treatment of acne, comprising topically applying onto an affected skin area of a subject in need thereof, once a day for a period of time of at least about 2, 4, 8, or 12 weeks, a topical medicament which comprises tretinoin or a pharmaceutically acceptable salt thereof, as a single active agent in an amount of between about 0.01% to about 0.5% weight; wherein said active agent is encapsulated in a shell; and

wherein the score of at least one parameter evaluated by an Investigator Cutaneous Safety Assessment is lower than the score of the parameters evaluated with the same treatment regimen using a non-encapsulated active agent. In another embodiment, the topical medicament comprises about 0.1% w/w of tretinoin and about 3% w/w of benzoyl peroxide.

In another one of its aspects, the present invention provides a regimen for providing early onset of action in the treatment of acne, comprising topically applying onto an affected skin area of a subject in need thereof, once a day for a period of time of at least about 2, 4, 8, or 12 weeks, a topical medicament which comprises tretinoin or a pharmaceutically acceptable salt thereof, as a single active agent in an amount of between about 0.01% to about 0.5% weight; wherein said active agent is encapsulated in a shell; and

wherein the score of at least one parameter evaluated by an Investigator Cutaneous Safety Assessment is lower than the score of the parameters evaluated with the same treatment regimen using a non-encapsulated active agent. In another embodiment, the topical medicament further comprises about 0.1% w/w of tretinoin and about 3% w/w of benzoyl peroxide.

In a further aspect, the present invention provides a regimen for the treatment of acne, comprising topically applying onto an affected skin area of a subject in need thereof, once a day for a period of time of up to 12 weeks, a topical medicament which comprises tretinoin or a pharmaceutically acceptable salt thereof, as a single active agent in an amount of between about 0.05% to about 0.1% weight; wherein said active agent is encapsulated in a shell; and wherein the score of at least one parameter evaluated by Local Tolerability Score is lower than the score of the parameters evaluated with the same treatment regimen using a non-encapsulated active agent. In another embodiment, the topical medicament comprises about 0.1% w/w of tretinoin and about 3% w/w of benzoyl peroxide.

In some embodiments, the present invention provides a regimen for the treatment of acne, comprising topically applying onto an affected skin area of a subject in need thereof, once a day for a period of time of about at least about 2, 4, 8, 12 weeks, a topical medicament which comprises tretinoin or a pharmaceutically acceptable salt thereof, as a single active agent in an amount of between about 0.05% to about 0.1% weight; wherein said active agent is encapsulated in a shell; and

wherein the score of at least one parameter evaluated by Local Tolerability Score is lower than the score of the parameters evaluated with the same treatment regimen using a non-encapsulated active agent. In another embodiment, the topical medicament comprises about 0.1% w/w of tretinoin and about 3% w/w of benzoyl peroxide.

In some embodiments, the present invention provides a regimen for the treatment of acne, comprising topically applying onto an affected skin area of a subject in need thereof, once a day for a period of time of about at least about 2, 4, 8, 12 weeks, a topical medicament which comprises tretinoin or a pharmaceutically acceptable salt thereof, as a single active agent in an amount of between about 0.01% to about 0.5% weight; wherein said active agent is encapsulated in a shell; and

wherein the score of at least one parameter evaluated by Local Tolerability Score is lower than the score of the parameters evaluated with the same treatment regimen using a non-encapsulated active agent. In another embodiment, the topical medicament further comprises about 0.1% tretinoin and about 3% benzoyl peroxide.

In some embodiments, the present invention provides a regimen for early onset for the treatment of acne, wherein the regimen comprises topically applying onto an affected skin area of a subject in need thereof, once a day for a period of time of about at least about 2, 4, 8, 12 weeks, a topical medicament which comprises tretinoin or a pharmaceutically acceptable salt thereof, as a single active agent in an amount of between about 0.01% to about 0.5% weight; wherein said active agent is encapsulated in a shell; and

wherein the score of at least one parameter evaluated by Local Tolerability Score is lower than the score of the parameters evaluated with the same treatment regimen using a non-encapsulated active agent. In another embodiment, the topical medicament comprises about 0.1% w/w of tretinoin and about 3% w/w of benzoyl peroxide.

In some embodiments, the amount of said encapsulated tretinoin is about 0.1% weight.

In another embodiment, the present invention provides a regimen for the treatment of acne comprising: topically applying onto an affected skin area of a subject in need thereof, once a day for a period of time of up to 12 weeks, a topical medicament which comprises the active agents:
tretinoin or a pharmaceutically acceptable salt thereof, in an amount of between about 0.05% to about 0.1% weight; and
benzoyl peroxide in an amount of at least about 3% weight;

wherein said regimen reduces at least one of:

(i) the number of inflammatory acne lesions by at least 50%; or

(ii) the number of non-inflammatory acne lesions by at least 40%.

In another embodiment, the present invention provides a regimen for early onset for the treatment of acne, wherein the regimen comprises topically applying onto an affected skin area of a subject in need thereof, once a day for a period of time of at least about 2, 4, 8, or 12 weeks, a topical medicament which comprises the active agents:
tretinoin or a pharmaceutically acceptable salt thereof, in an amount of between about 0.05% to about 0.1% weight; and
benzoyl peroxide in an amount of at least about 3% weight;

wherein said regimen reduces at least one of:

(i) the number of inflammatory acne lesions by at least 50%; or

(ii) the number of non-inflammatory acne lesions by at least 40%.

In another embodiment, the present invention provides a regimen for early onset for the treatment of acne, wherein the regimen comprises topically applying onto an affected skin area of a subject in need thereof, once a day for a period of time of at least about 2, 4, 8, or 12 weeks, a topical medicament which comprises the active agents:
tretinoin or a pharmaceutically acceptable salt thereof, in an amount of between about 0.01% to about 0.5% weight; and
benzoyl peroxide in an amount of between about to about 0.5% weight;

wherein said regimen reduces at least one of:

(i) the number of inflammatory acne lesions by at least 50%; or

(ii) the number of non-inflammatory acne lesions by at least 40%.

In some embodiments, the acne being treated by the regimen provided herein is mild acne, moderate acne, or severe acne. In some exemplary embodiments, the subject has mild acne. In some exemplary embodiments, the subject has moderate acne. In some exemplary embodiments, the subject has severe acne. In some exemplary embodiments, the acne is facial acne. In some exemplary embodiments, the subject has severe acne as determined by Investigator Global Assessment (IGA).

In some embodiments, the amount of said tretinoin used in the regimen provided herein is about 0.1% weight and the amount of said benzoyl peroxide is at least about 3% weight. It should be noted that the composition having these active agents in these concentrations was shown to have unexpected and surprising benefits with respect to the tolerability of the product (less side effects such as burning and itching, stinging and so forth), safety of the treatment (less erythema, scaling, pigmentation and so forth), and effectiveness of the treatment of acne (treatment with the composition following the regimen of the invention significantly reduced the number of non-inflammatory and inflammatory acne lesions). Surprisingly, when increasing the concentration of the tretinoin from 0.05% to 0.1%, while the efficacy increased, the side effects were not increased and in some cases were even reduced. For example, 44.8% subjects complained about burning side effects at 12 weeks of using the composition comprising 0.05% tretinoin and 3% benzoyl peroxide (see Table 21), while only 38.1% subjects complained about burning side effect when increasing the concentration of the tretinoin to 0.1% (see Table 18).

In some embodiments the regimen provided herein surprisingly reduces the number of mean inflammatory acne lesions by at least 40% after only 4 weeks of treatment. In other embodiments, said regimen reduces the number of non-inflammatory acne lesions by at least 35% after only 4 weeks of treatment. In yet further embodiments, said regimen reduces the number of inflammatory acne lesions by at least 40%; and the number of non-inflammatory acne lesions by at least 35%.

In some embodiments, after two weeks storage at 40° C. of the topical medicament of the invention, the concentration of RRT (relative retention time) 0.44, (all-trans 5,6-epoxy retinoic acid, that is the major tretinoin degradation product) is lower than 1%. In other embodiments, after two weeks storage at 40° C. of the topical medicament of the invention, the degradation of said tretinoin is less than 2.5%.

In some embodiments, after two weeks storage at 40° C. of the pharmaceutical composition, the concentration of all-trans 5,6-epoxy retinoic acid can be lower than 1%.

In some embodiments, after two weeks storage at 40° C. of the pharmaceutical composition, the degradation of said tretinoin is less than 2.5%.

In some embodiments, the regimen potentiates the action of tretinoin in the treatment of acne.

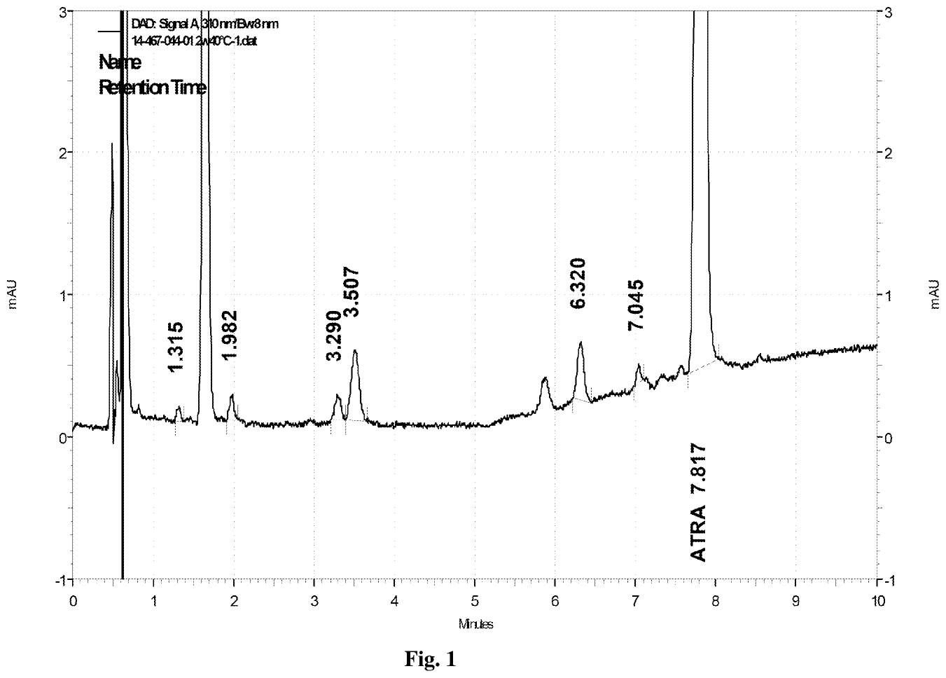

When referring to RRT 0.44 it should be understood to relate to the degradation product of tretinoin in the presence of BPO as shown in the HPLC chromatography of the composition of the invention after two weeks of storage at 40° C. An example of the RRT product can be seen in FIG. 1 at retention time 3.507 min. In other embodiments, RRT 0.44 refers to all-trans 5,6-epoxy retinoic acid represented by the following structure:

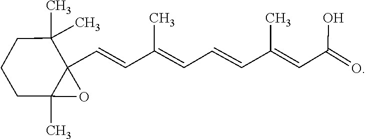 [SMILES: CC(/C=C/C12OC1(C)CCCC2(C)C)=C\C=C\C(C)=C\C(=O)O]

The invention further provides a method of treating acne, comprising topically applying onto an affected skin area of a subject in need thereof, once a day for a period of time of up to 12 weeks, a topical medicament which comprises of the active agents: tretinoin or a pharmaceutically acceptable salt thereof, in an amount of between about 0.05% to about 0.1% weight; and benzoyl peroxide in an amount of at least about 3% weight; wherein said method potentiates the action of tretinoin in the treatment of acne.

The present invention further provides a regimen for the treatment of acne comprising: topically applying onto an affected skin area of a subject in need thereof, once a day for a period of time of up to 12 weeks, a topical medicament which comprises the active agents: tretinoin or a pharmaceutically acceptable salt thereof, in an amount of between about 0.05% to about 0.1% weight; and benzoyl peroxide in an amount of at least about 3% weight; wherein the release rate (dissolution rate) of said tretinoin from said topical medicament is less than 60% per h. In some embodiments, the “release rate” (dissolution rate) of said tretinoin from said topical medicament is less than 50% per h. In some embodiments, the release rate (dissolution rate) of said tretinoin from said topical medicament is less than 45%, 40%, 35%, 30% or 25% per h.

The present invention further provides a regimen for the treatment of acne comprising topically applying onto an affected skin area of a subject in need thereof, once a day for a period of time of at least 2, 4, 8, or 12 weeks, a topical medicament which comprises the active agents: tretinoin or a pharmaceutically acceptable salt thereof, in an amount of between about 0.01% to about 0.5% weight; and benzoyl peroxide in an amount of between about 0.01% to about 0.5% weight; wherein the release rate (dissolution rate) of said tretinoin from said topical medicament is less than 60% per h. In some embodiments, the “release rate” (dissolution rate) of said tretinoin from said topical medicament is less than 50% per h. In some embodiments, the release rate (dissolution rate) of said tretinoin from said topical medicament is less than 45%, 40%, 35%, 30% or 25% per h.

It should be noted that the “release rate” (dissolution rate) defined herein relates to the measurement (either in vitro or in vivo) of the rate at which the active agents (for example tretinoin) is released from the topical medicament of the invention, to the extracting media or skin. The release rate is measured using known methods, such as for example: (1) 70% IPA (isopropyl alcohol) and 30% water and optionally an antioxidant (such as BHT) at room temperature; (2) 60-80% alcohol, ACN (acetonitrile) at room temperature; or (3) 2% Tween 80, IPA in a ratio of 2:1, and optionally an antioxidant (such as BHT) at 32° C.

In some embodiments, said release rate of said tretinoin from said topical medicament is less than 60% per h in a medium of 70% IPA (isopropyl alcohol) and 30% water at room temperature.

The invention further provides a regimen for the treatment of acne comprising topically applying onto an affected skin area of a subject in need thereof, once a day for a period of time of up to 12 weeks, a topical medicament which comprises the active agents:
tretinoin or a pharmaceutically acceptable salt thereof, in an amount of between about 0.05% to about 0.1% weight; and
benzoyl peroxide in an amount of at least about 3% weight;

wherein the score of at least one efficacy parameter is synergistically higher than the parameter evaluated with the same treatment regimen with each of the active agents separately. In another embodiment, the topical medicament comprises about 0.1% tretinoin and about 3% benzoyl peroxide.

In some embodiments, said efficacy parameter is selected from at least one of IGA success rate, mean reduction in inflammatory lesions count, mean reduction in non-inflammatory lesion count, mean reduction in acne symptom domain (measured using a patient reported outcome study and including symptoms such as for example number of pimples, whiteheads, blackheads, redness), mean reduction in acne impact domain (measured using a patient reported outcome study and including symptoms such as for example sadness, embarrassment, self-consciousness) and mean reduction in verbal rating scale.

As used herein the term “a regimen for the treatment of acne” is used herein interchangeably with the term “method of treating acne” having all the same meaning and qualities. The term “regimen” as used herein should be understood to relate to a medical treatment regimen regulating the treatment of acne in a subject suffering therefrom, including the regulation of the medicament administered (fixed dose combination of the active agents: tretinoin or a pharmaceutically acceptable salt thereof and BPO), the frequency of administration (i.e. once a day), the duration of treatment (i.e. up to 12 weeks), the method of administration (i.e. topical) and the location of administration (i.e. topically applying onto an affected skin area).

When relating to the treatment of “acne” it should be understood to relate to the treatment of a skin condition or disease also known as acne vulgaris in any form or place of its occurrence or severity (mild, moderate, severe or any combinations thereof. In some subjects parts of area of the skin may be mildly inflicted while other area of the skin of the same individual may be severely inflicted). Mild acne is classically defined as open (blackheads) and closed (whiteheads) clogged skin follicles (comedones) limited to the face with occasional inflammatory lesions. Acne may be considered to be of moderate severity when a higher number of inflammatory papules and pustules occur on the skin. Severe acne is said to occur when nodules are the characteristic facial lesions, and involvement of other areas of the body is extensive. Inflammatory acne lesions include papule lesions (small, solid elevation less than 5 mm in diameter, most of the lesion is above the surface of the skin), pustule lesions (small circumscribed elevation less than 5 mm in diameter that contains yellow-white exudate), nodule lesions (inflammatory lesion greater than or equal to 5 mm in diameter) and cyst lesions (inflammatory lesion that contains yellow-white exudate that is greater than or equal to 5 mm in diameter). Non-inflammatory lesions include open comedone (black head) (lesion in which the follicle opening is widely dilated with the contents protruding out onto the surface of the skin, with compacted melanin cells giving the plug a black appearance) and closed comedone (white head) (lesion in which the follicle opening is closed, but the sebaceous gland is enlarged by the pressure of the sebum buildup, which in turn cause the skin around the follicle to thin and become elevated with a white appearance).

As used herein, the term “treating” or “treatment” includes curing a condition, treating a condition, preventing or substantially preventing a condition, treating symptoms of a condition, curing symptoms of a condition, ameliorating, reducing and/or minimizing symptoms of a condition, treating effects of a condition, ameliorating, reducing and/or minimizing effects of a condition, and preventing and/or substantially preventing results of a condition,

As used herein, the term “topical use” is meant to encompass the topical administration of an exemplary composition by formulating said composition in any way known in the art, or in formulations disclosed herein, which are compatible with the skin, mucous membranes and/or the integuments.

As used herein, the term “early onset” or “early onset of action” means achieving a desired result and/or effect at a point in time that is earlier or even much earlier than achieved using a vehicle or other, conventional treatment approach. For example, it can mean achieving a desired result and/or effect no later than about 8 weeks from initial treatment, preferably no later than about 4 weeks from initial treatment, and more preferably no later than about 2 weeks from initial treatment.

As used herein, the term “pharmaceutical composition” refers to a composition comprising one or more active ingredients with other components such as, for example, pharmaceutically acceptable ingredients and/or excipients. The purpose of a pharmaceutical composition is to facilitate administration of an active ingredient to a subject. An example of a pharmaceutical composition is a topical medicament.

As used herein, the terms “pharmaceutically active agent” or “active agent” or “active pharmaceutical ingredient” are interchangeable and mean the ingredient is a pharmaceutical drug, which is biologically- and/or chemically active and is regulatory-approved or approvable as such.

As used herein, the term “ingredient” refers to a pharmaceutically acceptable ingredient, which is included or is amenable to be included in The FDA's Inactive Ingredient (IIG) database. Inactive ingredients can sometimes exhibit some therapeutic effects, although they are not drugs.

As used herein, the term “adverse events values” refers to an average percentage of subjects that experience any adverse events associated with the treatment of acne with a composition described and/or claimed herein (usually on a surface of the skin of a subject treated with a composition described and/or claimed herein). A non-limiting list of such adverse events includes erythema, pigmentation, irritation, dryness, scaling, itching, pruritus, burning, stinging, combinations thereof and the like.

The term “synergistically lower” as used herein should be understood to relate to the degree of lowering the side-effects (as measured using Investigator Cutaneous Safety Assessment and Local Tolerability Score) caused by topical administration of the active agents in a regimen of the invention, as compared with the sum of the side-effects resulting from administration of each of the agents separately.

The term “synergistically higher” as used herein should be understood to relate to the degree of therapeutic efficacy (as measured using efficacy results selected from at least one of IGA success rate, mean reduction in inflammatory lesions count, mean reduction in non-inflammatory lesion count; and/or PRO results selected from at least one of mean reduction in acne symptom domain, mean reduction in acne impact domain and mean reduction in verbal rating scale) caused by topical administration of the active agents in a regimen of the invention, as compared with the sum of the effect resulting from administration of each of the agents separately.

The synergistic lower side-effect of the regimen of the invention is calculated according to the following formula:

(TWIN-V)<(ATRA-V)+(BPO-V)

TWIN—side effect (using the score measurement indicated above and below) measured for the medicament defined in the present invention.

V—side effect (using the score measurement indicated above and below) measured for the vehicle alone.

ATRA—side effect (using the score measurement indicated above and below) measured for ATRA alone.

BPO—side effect (using the score measurement indicated above and below) measured for BPO alone.

When the effect of the medicament of the invention is measured, the side-effect of the vehicle (V) is subtracted from the side-effect of the medicament (TWIN), the net side-effect of the medicament of the invention (TWIN-V) is numerically lower than the sum of the net clinical benefits of each of the individual active agent after subtraction of the vehicle effect from the ATRA and BPO branches, respectively.

The synergistic higher effect of the regimen of the invention is calculated according to the following formula:

(TWIN-V)>(ATRA-V)+(BPO-V)
TWIN—therapeutic effect (using the score measurement indicated above and below) measured for the medicament defined in the present invention.
V—therapeutic effect (using the score measurement indicated above and below) measured for the vehicle alone.
ATRA—therapeutic effect (using the score measurement indicated above and below) measured for ATRA alone.
BPO—therapeutic effect (using the score measurement indicated above and below) measured for BPO alone.

When the effect of the medicament of the invention is measured, the therapeutic effect of the vehicle (V) is subtracted from the therapeutic effect of the medicament (TWIN), the net therapeutic effect of the medicament of the invention (TWIN-V) is numerically higher than the sum of the net clinical benefits of each of the individual active agent after subtraction of the vehicle effect from the ATRA and BPO branches, respectively.

It is to be noted that the effect of the regimen of the invention, wherein the two active agents are administered in combination is at least an additive effect and preferentially a synergistic effect. In some embodiments, the synergistic effect refers to the synergistic lowering of the side effects caused by administration of the active agents. In some other embodiments, the additive effect of the regimen of the invention is attributed to the clinical therapeutic effect of the active agents. In further embodiments, the synergistic effect of the regimen of the invention is attributed to the clinical therapeutic effect of the active agents.

It is further noted that any of the above synergistic effects can be attributed to the effect at—at least one of week 2, 4, 8, 12 of the regimen of the invention. In some embodiments, the synergistic effect is provided at week 2 of the regimen of the invention. In some embodiments, the synergistic effect is provided at week 4 of the regimen of the invention. In some embodiments, the synergistic effect is provided at week 8 of the regimen of the invention. In some embodiments, the synergistic effect is provided at week 12 of the regimen of the invention.

When referring to “improvement”, “improves” and any other lingual derivatives of the term it should be understood to include an additive or synergistic therapeutic effect of the regimen of the invention. When referring to “improvement of the IGA success rate by at least 20%” it should be understood to relate to an additive or, in some embodiment synergistic, improvement of the Investigational Global Assessment (IGA) success rate measured for degree of success in reducing the number of acne lesions and an improvement in the clinical condition of patients compared to their baseline condition/score.

The term “potentiates the action of tretinoin in the treatment of acne” should be understood to encompass any therapeutic augmentation of the treatment of acne achieved by administering tretinoin to a subject suffering from acne. The therapeutic effect of administering a topical medicament comprising both tretinoin and benzoyl peroxide is either additive or synergistic to the effect of acne treatment with tretinoin alone.

### Pharmaceutical Composition

In some embodiments, the regimen provided herein make use of a pharmaceutical composition comprising tretinoin or a pharmaceutically acceptable salt thereof, in an amount of about 0.01% to about 0.5% weight; and benzoyl peroxide in an amount of between about 1% to about 10% weight. In other embodiments, the composition comprises tretinoin or a pharmaceutically acceptable salt thereof, in an amount of about 0.05% to about 0.1% weight; and benzoyl peroxide in an amount of between about 1% to about 10% weight.

In another embodiment, the topical medicament comprises about 0.1% w/w of tretinoin and about 3% w/w of benzoyl peroxide.

In some embodiments the pharmaceutical composition used in the regimen of this invention comprises benzoyl peroxide in an amount of about 1% weight. In some embodiments, the pharmaceutical composition comprises about 2% w/w to about 10% w/w of benzoyl peroxide, about 2% w/w to about 6% w/w of benzoyl peroxide. In some embodiments, the pharmaceutical composition comprises about 3% w/w to about 6% w/w of benzoyl peroxide. In some embodiments of regimen of the present invention, said benzoyl peroxide is in an amount of about 3% weight. In other embodiments of regimen of the present invention, said benzoyl peroxide is in an amount of about 6% weight. In other embodiments of regimen of the present invention, said benzoyl peroxide is in an amount of about 10% weight. In further embodiments of regimen of the present invention, said benzoyl peroxide is in an amount of between about 1% to about 10% weight. In further embodiments of regimen of the present invention, said benzoyl peroxide is in an amount of between about 3% to about 6% weight. In yet further embodiments of regimen of the present invention, said benzoyl peroxide is in an amount of about 1%, about 2%, about 3%, about 4%, about 5%, about 6%, about 7%, about 8%, about 9% or about 10% weight. In another embodiment, the pharmaceutical composition comprises about 3% w/w of benzoyl peroxide.

In some embodiments, the pharmaceutical composition used in the regimen of this invention comprises about 0.01% w/w to about 0.5% w/w of tretinoin or a pharmaceutically acceptable salt thereof. In another embodiment, the pharmaceutical composition comprises about 0.05% w/w to about 0.1% w/w of tretinoin or a pharmaceutically acceptable salt thereof. In another embodiment, the pharmaceutical composition comprises about 0.05% w/w to about 0.2% w/w of tretinoin or a pharmaceutically acceptable salt thereof. In another embodiment, the pharmaceutical composition comprises about 0.01% w/w to about 0.1% w/w of tretinoin or a pharmaceutically acceptable salt thereof. In another embodiment, the pharmaceutical composition comprises about 0.1% w/w of tretinoin or a pharmaceutically acceptable salt thereof.

In some embodiments, said amount of said tretinoin or said pharmaceutically acceptable salt thereof, is at least 0.01% weight.

In some embodiments, said amount of said tretinoin or said pharmaceutically acceptable salt thereof, is at least 0.05% weight.

In other embodiments, said amount of said tretinoin or said pharmaceutically acceptable salt thereof, is about 0.1% weight. In some embodiments, said amount of said tretinoin or said pharmaceutically acceptable salt thereof, is about 0.5% weight.

In some embodiments, said amount of said tretinoin or said pharmaceutically acceptable salt thereof, is about 0.01% to about 0.5% weight.

In some embodiments, said amount of said tretinoin or said pharmaceutically acceptable salt thereof, is about 0.05% to about 0.1% weight.

In another embodiment, the topical medicament comprises about 0.1% w/w of tretinoin and about 3% w/w of benzoyl peroxide. In another embodiment, the topical medicament used in the regimen treatment or method of treatment of this invention is described in US patent publication 2013/0095185, which is incorporated herein by reference.

In some embodiments, the benzoyl peroxide and the tretinoin or a pharmaceutically acceptable salt thereof are the sole active ingredients administered to the subject in need of said treatment during the duration of the regimen.

In some embodiments the pharmaceutical composition comprises tretinoin or a pharmaceutically acceptable salt, hydrate or solvate.

In some embodiments, the pharmaceutical composition is free or substantially free of acetone.

In some exemplary embodiments, the pharmaceutical composition is a topical medicament.

In some exemplary embodiments, the pharmaceutical composition is a cream or an emulsion.

In some embodiments, the pharmaceutical composition is applied to the face.

In some exemplary embodiments, the pharmaceutical composition is an extended-release formulation. The extended-release effect can be obtained by encapsulation, microencapsulation, microspheres, coating, a combination thereof, or the like. In some embodiments, the benzoyl peroxide is encapsulated and/or microencapsulated. In some embodiments the benzoyl peroxide is included in a microsphere, a coating, a combination thereof, or the like. In some embodiments, the tretinoin or a pharmaceutically acceptable salt thereof is encapsulated and/or microencapsulated, in some embodiments the tretinoin or a pharmaceutically acceptable salt thereof is included in a microsphere, a coating, a combination thereof, or the like.

In some embodiments, the benzoyl peroxide is in a form selected from solid or suspension. In another embodiment, the benzoyl peroxide is in a solid form. In another embodiment, the benzoyl peroxide is encapsulated and/or microencapsulated.

In another embodiment, benzoyl peroxide is included in a microsphere and/or a coating. In another embodiment, the tretinoin or a pharmaceutically acceptable salt thereof is encapsulated and/or microencapsulated. In another embodiment, the tretinoin or a pharmaceutically acceptable salt thereof is included in a microsphere and/or a coating. In another embodiment, at least one active ingredient of the pharmaceutical composition is encapsulated in a shell. In another embodiment, the benzoyl peroxide and tretinoin, of the pharmaceutical composition is encapsulated in its own, separate shell. In another embodiment, the shell is an inorganic shell. In another embodiment, the shell is a metal oxide or semi-metal oxide inorganic shell.

In another embodiment, the pharmaceutical composition provided herein is a single dose composition comprising both said active agents.

In another embodiment, the pharmaceutical composition provided herein, further comprises at least one nonpharmaceutical active additive selected from the group consisting of chelating agents, antioxidants, sunscreens, preservatives, fillers, electrolytes, humectants, dyes, mineral or organic acids or bases, fragrances, essential oils, moisturizers, vitamins, essential fatty acids, sphingolipids, self-tanning compounds, calmatives and skin-protecting agents, pro-penetrating agents and gelling agents, or a mixture and/or combination thereof. In another embodiment, the pharmaceutical composition provided herein, comprises about 2% w/w to about 10% w/w of benzoyl peroxide.

In some embodiments, the pharmaceutically acceptable salt of tretinoin is selected from ammonium, lithium, sodium, potassium, cesium; alkaline earth metals to include calcium, magnesium, aluminium; zinc, barium; or quaternary ammoniums; or organic salts such as arginine, organic amines to include aliphatic organic amines, aromatic amines, t-butylamines, (N-benzylphenethylamine), dicyclohexylamines, dimethylamines, diethanolamines, ethanolamines, ethylenediamines, imidazoles, lysines, methylamines, N-methyl-D-glucamines, N,N′-dibenzylethylenediatnines, pyridines, picolinates, piperazines, tris(hydroxymethyl)methylamines, triethylamines, triethanolamines, trimethylamines, or ureas.

In some embodiments, the tretinoin or a pharmaceutically acceptable salt thereof is in a form selected from solid, solution or suspension. In another embodiment, the tretinoin or a pharmaceutically acceptable salt thereof is in a solid form.

In some embodiments, said medicament is applied at least twice a day. In some further embodiments, said medicament is applied once a day. In some further embodiments, said medicament is applied twice a day. In other embodiments, said medicament is applied twice a day with a period of at least 8 hours between administrations. In some embodiments, said medicament is applied every other day.

In some embodiments, said medicament is administered for a period of up to 12 weeks. In some embodiments, said medicament is administered for a period of 12 weeks. In other embodiments, said medicament is administered for a period of 1 week. In some embodiments, said medicament is administered for a period selected from 1, 2, 4, 8 and 12 weeks. In some embodiments, said medicament is administered for a period of 2 weeks. In some embodiments, said medicament is administered for a period of 4 weeks. In some embodiments, said medicament is administered for a period of 8 weeks. In some embodiments, said medicament is administered for a period of 12 weeks.

In some embodiments, said medicament is administered in a single composition, single fixed dose medicament, comprising both said active agents (BPO and ATRA). In such embodiments, the weight % of the active agent relates to their weight amount in the single composition. The term “fixed dose medicament” should be understood as meaning a combination whose active agents are combined at fixed doses in the same vehicle (single formula) that delivers them together to the point of application.

In further embodiments, said medicament comprises two separate compositions each one comprising each of said active agents. In such embodiments, the weight % amount of each active agent relates to each of their weight amount in each composition separately. In some embodiments, said two separate compositions of said medicament are administered concomitantly. In further embodiments, said two separate compositions are administered sequentially.

In some embodiments, at least one of said active agents in a medicament disclosed hereinabove is encapsulated in a shell. In some other embodiments, both active agents, benzoyl peroxide (BPO) and tretinoin, of said medicament are encapsulated in a shell. In some embodiments, each of the active agents, benzoyl peroxide and tretinoin, of the pharmaceutical composition is encapsulated in its own, separate shell. Encapsulation of active agents in separate shells prevent or substantially minimize cross-reactivity. In some embodiments, said shell is an inorganic shell. In further embodiments, said encapsulating shell is a metal oxide or semi-metal oxide inorganic shell.

In some embodiments, the pharmaceutical composition is a single dose composition comprising both said active agents.

In some embodiments, the pharmaceutical composition further comprises at least one nonpharmaceutical active additive selected from the group consisting of chelating agents, antioxidants, sunscreens, preservatives, fillers, electrolytes, humectants, dyes, mineral or organic acids or bases, fragrances, essential oils, moisturizers, vitamins, essential fatty acids, sphingolipids, self-tanning compounds, calmatives and skin-protecting agents, pro-penetrating agents and gelling agents, or a mixture and/or combination thereof.

In some embodiments, the release rate of said tretinoin from said pharmaceutical composition can be less than 60% per h. In some embodiments, the release rate of said tretinoin from said pharmaceutical composition can be less than 60% per h in a medium of 7.0% IPA (isopropyl alcohol) and 30% water at room temperature.

In some embodiments, the pharmaceutical composition is applied once daily for a period of about 2, 4, 8 or 12 weeks. In some exemplary embodiments, the pharmaceutical composition is applied once daily for a period of about 4, 8 or 12 weeks. In some exemplary embodiments, the pharmaceutical composition is applied once daily for a period of about 2 weeks. In some exemplary embodiments, the pharmaceutical composition is applied once daily for a period of about 4 weeks. In some embodiments, the pharmaceutical composition is applied once daily for a period of about 8 weeks. In some exemplary embodiments, the pharmaceutical composition is applied once daily for a period of about 12 weeks.

As used herein unless otherwise indicated the term “microcapsule” refers to a microparticle having a core shell structure, wherein said core comprises an active agent as defined herein (core material), being coated by a shell forming the microcapsule entrapping the core. In some embodiments, the coating/shell is directly deposited on the core material. In some embodiments, the core material is solid. In other embodiments, the core material is semi-solid. In some embodiments, the core material consists of a solid particle of the active agent. In other embodiments, the core material comprises a solid particle of the active agent. In some other embodiments, the core material is in a liquid/oily phase.

The size of the microcapsules (denoted herein also by the general term “particles” or “microparticles”) as will be referred to herein refers to D₉₀ meaning that 90% of the particles have the stated dimension or less (measured by volume). Thus, for examples, for spherical particles stated to have a diameter of 10 micrometers (“microns”), this means that the particles have a D₉₀ of 10 microns. The D₉₀ (termed also d(0.9)) may be measured by laser diffraction. For particles having a shape other than spheres, the D₉₀ refers to the mean average of the diameter of a plurality of particles.

In some embodiments, the microcapsules are formed using the process as disclosed in the following documents (herein incorporated by reference): U.S. Pat. Nos. 6,303,149, 6,238,650, 6,468,509, 6,436,375, US2005037087, US2002064541, and International publications Nos. WO 00/09652, WO00/72806, WO 01/80823, WO 03/03497, WO 03/039510, WO00/71084, WO05/009604, and WO04/81222, disclose sol-gel microcapsules and methods for their preparation; EP 0 934 773 and U.S. Pat. No. 6,337,089 teach microcapsules containing core material and a capsule wall made of organopolysiloxane, and their production; EP0941 761 and U.S. Pat. No. 6,251,313 also teach the preparation of microcapsules having shell walls of organopolysiloxane; U.S. Pat. No. 4,931,362 describes a method of forming microcapsules or micromatrix bodies having an interior water-immiscible liquid phase containing an active, water-immiscible ingredient. Microcapsules prepared by a sol-gel process are also disclosed in GB2416524, U.S. Pat. No. 6,855,335, WO03/066209.

According to some embodiments of the present invention, the coated form of the active ingredient (microcapsule) may be in form of a polymeric microsponge/silica microsphere where the active ingredient is adsorbed, embedded, impregnated or entrapped in the microsponge/silica microsphere as described for example in U.S. Pat. Nos. 4,690,825; 5,145,675, 5,879,716, 5,955,109, and 9,452,137 incorporated herein by reference in their entirety.

In other embodiments, microcapsules are formed by the encapsulation process disclosed in the following publications (herein incorporated by reference): U.S. Pat. Nos. 7,629,394, 9,205,395, US 2015/0328615, US 2014/0186630. Controlled release microcapsules: IN01958CH2007, IN02080CH2007, U.S. Pat. Nos. 4,235,872, 4,670,250, EP 0248531, U.S. Pat. Nos. 4,970,031, 5,238,714, WO9321764, U.S. Pat. No. 5,575,987, WO9420075, US 2004/137031, US 2006/003014, US 2010/180464.

The core (wherein it is a solid particulate matter) may be of any shape for example rod-like, plate-like, ellipsoidal, cubic, or spherical shape.

In the case of cores having a spherical shape, the diameter (D₉₀) may be in the range of 0.3 to 90 microns, in some embodiments 0.3 to 50 microns, in some further embodiments 1 to 50, in some further embodiments 5 to 30 microns.

By the term “the diameter (D₉₀) may be in the range of 0.3 to 90 microns” is meant that 90% by volume of the particles (in this case the particle's core) may be less than or equal to a value in the range of 0.3 to 90 microns.

For generally cubic-shaped cores or cores having a shape resembling that of a cube, the mean size of a side may be in the range 0.3 to 80 microns, in some embodiments 0.3 to 40 microns, in some further embodiments 0.8 to 40 microns, in some further embodiments 4 to 15 microns.

For rod-like shaped, ellipsoidal-shaped and plate-like shaped cores, the largest dimension (that of the longest axis) is typically in the range 10 to 100 microns, in some embodiments 15 to 50 microns; and the smallest dimension is typically in the range 0.5 to 20 microns and in some further embodiments 2 to 10 microns.

According to an embodiment of the present invention, the microcapsules (coated particulate matter) have a diameter (d90) of 0.5 to 100 μm or in some embodiments the diameter of the microcapsules is in the range of 1 to 50 μm and in some other embodiments in the range of 5 to 30 μm. It is appreciated that the microcapsules of the present invention are composed of distinct regions of the metal oxide layer in the core material (i.e. the water insoluble particulate matter).

Further according to an embodiment of the present invention the obtained metal oxide coating layer has a width (thickness) of 0.1 μm or above, in some embodiments the metal oxide coating layer has a width (thickness) of 0.1-10 μm.

Additionally, according to an embodiment of the present invention the obtained metal oxide coating layer has a width (thickness) of 0.3 μm or above, in some embodiments the metal oxide coating layer has a width of 0.3-10 μm.

Additionally, according to an embodiment of the present invention, the thickness of the metal oxide layer is in the range of 0.1-10 μm. In some further embodiments, the thickness of the metal oxide layer is in the range of 0.1-3 μm, and in some further embodiments in the range of 0.1-1 μm. The thickness of the metal oxide layer may also be in some embodiments in the range of 0.3 to 3 μm, and in some other embodiments in the range of 0.3 to 2 μm.

Further according to an embodiment of the present invention the obtained metal oxide coating layer has a width (thickness) of about 0.1, 0.2, 0.3, 0.5, 0.7, 1, 1.5, 2 or 5 μm or above, in some embodiments up to 10 μm.

The width of the metal oxide layer may be determined for example by a Transmission Electron Microscope or Confocal Microscope such that in a circular cross-sectional area of the particle the smallest width is at least e.g. 0.1 μm (the width is determined as the smallest distance from the surface of the particle (i.e. metal oxide surface) to the core-metal oxide interface).

The microcapsules are in some embodiments characterized in that the core material is substantially free of the metal oxide and further in that the metal oxide layer is substantially free of the core material, e.g. either as particle dispersion (in the nano-metric range of below 0.1 μm) of the particulate matter or as molecular dispersion of the particulate matter.

Thus, according to an embodiment of the present invention, the metal oxide layer is substantially free of core material (either in the form of molecules or as nano-metric particles). The term “substantially free” in this context denotes that the concentration of the molecules of the core material or the concentration of the nano-metric particles of the core material is negligible as compared to the metal oxide. Similarly, by the term “the core material is substantially free of the metal oxide” is meant that the concentration of the metal oxide in the core is negligible as compared to the core material. The microcapsules (i.e. first microcapsules) are in some embodiments non leaching when dispersed in a carrier and in some other embodiments non leaching in an aqueous based carrier.

According to another embodiment when the microcapsules are prepared by a method such as spray drying, the core material comprising the active agent may further comprise up to about 30% w/w, in some embodiments up to about 20% metal oxide and the metal oxide coating layer may further comprise up to about 30% w/w, in some embodiments up to about 20% w/w of the active agent.

By the term “non-leaching” it is meant that the leaching of the particulate matter (active agent) from the particles into an aqueous-based liquid is less than 5% w/w, in some embodiments less than 3%, in some further embodiments less than 1% w/w in some further embodiments less than 0.5% w/w, and in some other embodiments less than 0.1% w/w at room temperature (20° C.), under gentle agitation for 1 hour or until a steady state concentration is achieved. Typically, the aqueous-based liquid is water. The values indicated above refer to the percentage of the active agent leached into an aqueous medium relative to the initial amount of the active agent in the particles. The leaching values indicated above refer in some embodiments to a dispersion having a concentration of the particulate matter in the aqueous medium higher than 0.1% w/w, in some further embodiments higher than 1% w/w, in some further embodiments higher than 3% w/w, and in some other embodiments higher than 10% w/w. For tretinoin the leaching values indicated above refer in some embodiments to a dispersion having a concentration of the particulate matter in the aqueous medium higher than 0.01% w/w.

According to an embodiment of the present invention the weight ratio of the metal oxide to the solid particulate matter is in the range of 1:99 to 50:50. The weight ratio of the metal oxide layer to the solid particulate matter may be also in the range of 3:97 to 50:50, 5:95 to 50:50, 10:90 to 50:50, 5:95 to 30:70, 10:90 to 30:70. Further, according to an embodiment of the present invention the rate ratio of the metal oxide to the solid particulate matter is in the range of 10:90 to 20:80.

According to another embodiment of the present invention, when spray drying method is used, the weight ratio of the metal oxide to the solid particulate matter may be in the range 5:95 to 95:5.

As used herein by the term “uncoated free form” is meant that the active ingredient (BPO or tretinoin) is present in the composition in its “naked” form meaning that it is not intimately embedded, encapsulated, entrapped or encased in a polymeric carrier, and is present in the composition in direct contact with the composition carrier. As used herein by the term “coated form of the active ingredient” is meant that the active ingredient is embedded, dispersed, entrapped, or encased, e.g. as a solid dispersion or molecular dispersion in a polymeric carrier which may be an organic or inorganic carrier and which may serve as a matrix for dispersing the active ingredient or as encapsulated material coating said active ingredient (i.e. the active ingredient is present in a core or is a core material encapsulated by a shell composed of a polymeric material which may be an organic or inorganic polymer).

According to another embodiment of the present invention, the coated form of the active ingredient is second microcapsules comprising a solid particulate matter of the active ingredient coated by a metal oxide layer.

Further, according to an embodiment of the present invention, the first microcapsules comprise a solid particulate matter of BPO coated by a metal oxide layer.

According to an embodiment of the present invention, the BPO is in the form of first microcapsules comprising solid particulate matter of BPO coated by a metal oxide layer and the tretinoin is in the form of second microcapsules comprising a solid particulate matter of the tretinoin coated by a metal oxide layer.

Under these embodiments, the metal oxide coating layer is advantageous since it is capable of isolating the particulate matter of the active agent from its surrounding medium, thus preventing cross-reactivity of the active agents present in the same composition and yet enables the release the particulate matter upon application to the surface to be treated.

The term “solid water insoluble agent” refers to a solid material having solubility in water of less than 3% w/w, typically less than 1% and at times less than 0.5% w/w at room temperature (20° C.). The “solid water insoluble agent” may have a solubility of less than 0.1% w/w.

The “solid water insoluble agent” may also be termed herein as “solid water insoluble particulate matter” or “solid particulate matter”.

The term “topical medicament” as used herein (also referred to as “composition”) should be understood to encompass any pharmaceutical formulation that enables the administration of the active agents to a skin tissue of a patient administered with said medicament. The composition or topical medicament of the present invention comprises a carrier. According to an embodiment of the present invention the carrier is in the form of an ointment, a cream, a lotion, an oil, a solution (in some embodiments an aqueous solution), an emulsion, a gel, a paste, a milk, an aerosol, a powder, or a foam. In some embodiments the carrier is an aqueous-based carrier (such as a gel, oil-in water emulsion or oil-in water cream, aqueous solution, foam, lotion, spray).

Thus, the final form of the composition may be any of the above forms, mentioned with respect to the carrier, where the microcapsules are dispersed in the carrier. The final form of the composition may also be in the form of a wash or cleanser.

In some embodiments, the metal oxide is selected from silica, titania, alumina, zirconia, ZnO, and mixtures thereof. In some other embodiments the metal oxide is silica.

Moreover, according to an embodiment of the present invention, the microcapsules (coated particulate matter) have a diameter of 0.5-100 μm. In some embodiments, the particles have a diameter of 0.8-100 μm, in some further embodiments 1-50 μm and in some other embodiments 2-30 μm.

According to certain embodiments of the present invention, the surface of the metal oxide later of the coated particulate matter may be chemically modified by organic groups, in some embodiments hydrophobic groups, attached to its surface.

The hydrophobic groups may be for example an alkyl groups (such alkyl groups may be further substituted with one or more fluoro atoms), aryl groups (such as benzyl or phenyl), and combinations thereof. The groups may be as described below with respect to the process.

In some embodiments the topical medicament comprises tretinoin or its pharmaceutically acceptable salt, hydrate or solvate. Suitable pharmaceutically acceptable salts of the active component(s) (i.e. tretinoin) of this invention include inorganic salts such as: ammonium, alkali metals to include lithium, sodium, potassium, cesium; alkaline earth metals to include calcium, magnesium, aluminium; zinc, barium; or quaternary ammoniums; or organic salts such as arginine, organic amines to include aliphatic organic amines, aromatic amines, t-butylamines, (N-benzylphenethylamine), dicyclohexylamines, dimethylamines, diethanolamines, ethanolamines, ethylenediamines, imidazoles, lysines, methylamines, N-methyl-D-glucamines, N,N′-dibenzylethylenediamines, pyridines, picolinates, piperazines, tris(hydroxymethyl)methylamines, triethylamines, triethanolamines, trimethylamines, or ureas.

In one embodiment, the term “about”, refers to a deviance of between 0.0001-5% from the indicated number or range of numbers. In one embodiment, the term “about”, refers to a deviance of between 1-10% from the indicated number or range of numbers.

As used herein, the singular form “a,” “an” and “the” include plural references unless the context clearly dictates otherwise. For example, the term “a compound” or “at least one compound” can include a plurality of compounds, including combinations and/or mixtures thereof.

As used herein, the term “method” refers to manners, means, techniques and procedures for accomplishing a given task including, but not limited to, those manners, means, technical and procedures either known to, or readily developed from known manners, means, techniques and procedures by practitioners of the chemical, pharmacological, biological, biochemical and medical arts.

It is appreciated that certain features of the exemplary embodiments described herein, which are, for clarity, described in the context of separate embodiments, can also be provided in combination in a single embodiment. Conversely, various features of the exemplary embodiments, which are, for brevity, described in the context of a single embodiment, can also be provided separately or in any suitable sub-combination or as suitable in any other described embodiment. Certain features described in the context of various embodiments are not to be considered essential features of those embodiments, unless the embodiment is inoperative without those elements.

The following examples are presented in order to more fully illustrate the preferred embodiments of the invention. They should in no way, however, be construed as limiting the broad scope of the invention.

### EXAMPLES

### Example 1

Product 3149 is a combination formulation of encapsulated benzoyl peroxide (E-BPO) 3%, prepared as described in US patent publication 2010-0016443, and encapsulated All Trans Retinoic Acid (E-ATRA) 0.1%, prepared as described in US patent publication US 2012/0202695.

Product 3156 is a combination formulation of similar E-BPO 3% and similar E-ATRA 0.05%.

A clinical trial was designed to assess whether the combination of E-BPO and E-ATRA provides safe and synergistic efficacy as compared to use of either product alone in the treatment of acne vulgaris. Products 3149 and 3156 provide improved outcomes due the stability of the product, as described in US patent publication 2013/0095185, and lead to increased patient compliance.

The purpose of the study was to assess the efficacy, safety, and tolerability of Products 3149 and 3156 in comparison to the individual components, E-BPO 3%, E-ATRA 0.1%, E-ATRA 0.05%, and vehicle (placebo).

This was a randomized, double-blind, multicenter, parallel group, active- and vehicle-controlled study of the efficacy, tolerability, and safety of Products 3149 and 3156 for the treatment of acne vulgaris.

Approximately 720 subjects, age 9 and older, with moderate to severe facial acne (rated 3 or 4 on the 5-point Investigator's Global Assessment [IGA]) were enrolled at up to 37 sites. Participants were randomized 1:1:1:1:1:1 to receive once daily treatment with E-BPO/E-ATRA (3%/0.05%); E-BPO/E-ATRA (3%/0.1%); E-BPO (3%); E-ATRA (0.05%); E-ATRA (0.1%); and vehicle cream. After the screening period, qualified subjects were randomized at the Baseline visit and treated for 12 weeks.

Efficacy assessments included facial lesion counts (inflammatory and non-inflammatory) and IGA assessment ranging from 0 (Clear) to 4 (Severe). Investigators were provided with instructions for lesion counts to ensure consistency of procedure. Patient reported outcomes (PRO) were assessed at Baseline, Weeks 4, 8, and 12 or early termination. Safety was assessed at all visits and included monitoring local and systemic adverse experiences; the Investigator Cutaneous Safety Assessment rating of hyper- and hypopigmentation, erythema and scaling on a scale ranging from 0 (None) to 3 (Severe); and the subject assessment of Local Tolerability rating itching, burning, and stinging on a scale ranging from 0 (None) to 3 (Severe).

Subjects returned to centers for cutaneous safety and local tolerability assessments at Weeks 2, 4, 8, and 12; and IGA and lesion counts were repeated at Weeks 4, 8, and 12. Adverse events and concomitant medications were assessed throughout the treatment period.

All products were supplied in 80-gram pumps (50 g of it is the cream). One pea sized amount was applied on each area of the face (chin, left cheek, right cheek, nose, and forehead) once daily, at bedtime, for 12 weeks.

TABLE 1
Investigator's Global Assessment Scale for Acne Severity

| Score | Grade | Description |
|---|---|---|
| 0 | Clear | Normal, clear skin with no evidence of acne vulgaris |
| 1 | Almost | Rare non-inflammatory lesions present, with rare |
| | clear | non-inflamedpapules (papules must be resolving |
| | | and may be hyperpigmented, though not pink-red) |
| 2 | Mild | Some non-inflammatory lesions are present, with |
| | | few inflammatory lesions (papules/pustules only; |
| | | no nodulo-cystic lesions) |
| 3 | Moderate | Multiple Non-inflammatory lesions and, |
| | | inflammatory lesions are evident: several to many |
| | | comedones and papules/pustules, and there may |
| | | or may not be one small nodulo-cystic lesion |
| 4 | Severe | Inflammatory lesions are more apparent, many |
| | | comedones and papules/pustules, there may or may |
| | | not be a few nodulo-cystic lesions |

### Inflammatory Lesions Definitions

Papule: A small, solid elevation less than 5 mm in diameter. Most of the lesion is above the surface of the skin.

Pustule: A small circumscribed elevation less than 5 mm in diameter that contains yellow-white exudate.

Nodule: An inflammatory lesion greater than or equal to 5 mm in diameter.

Cyst: An inflammatory lesion that contains yellow-white exudate that is greater than or equal to 5 mm in diameter.

### Non-Inflammatory Lesions Definition

Open Comedone (Black head): A lesion in which the follicle opening is widely dilated with the contents protruding out onto the surface of the skin, with compacted melanin cells giving the plug a black appearance.

Closed Comedone (White head): A lesion in which the follicle opening is closed, but the sebaceous gland is enlarged by the pressure of the sebum buildup, which in turn cause the skin around the follicle to thin and become elevated with a white appearance.

TABLE 2
Cutaneous Safety Assessment (Investigator)

| | | |
|---|---|---|
| Score | Rating | Definition |

Erythema

| | | |
|---|---|---|
| 0 | None | No erythema |
| 1 | Mild | Slight pinkness present |
| 2 | Moderate | Definite redness, easily recognized |
| 3 | Severe | Intense redness |

Scaling

| | | |
|---|---|---|
| 0 | None | No scaling |
| 1 | Mild | Barely perceptible shedding, noticeable only on light |
| | | scratching or rubbing |
| 2 | Moderate | Obvious but not profuse shedding |
| 3 | Severe | Heavy scale production |

Pigmentation

| | | |
|---|---|---|
| 0 | None | No disturbance of pigmentation |
| 1 | Mild | Barely perceptible pigment change |
| 2 | Moderate | Markedly darker or lighter |
| 3 | Severe | Complete de-pigmentation or severe hyperpigmentation |

TABLE 3
Local Tolerability Scoring (Subject)

| | | |
|---|---|---|
| Score | Rating | Definition |

Itching

| | | |
|---|---|---|
| 0 | None | No itching |
| 1 | Mild | Slight itching, not really bothersome |
| 2 | Moderate | Definite itching that is somewhat bothersome |
| 3 | Severe | Intense itching that may interrupt daily |
| | | activities and/or sleep |

Burning

| | | |
|---|---|---|
| 0 | None | No burning |
| 1 | Mild | Slight burning sensation; not really bothersome |
| 2 | Moderate | Definite warm, burning sensation that |
| | | is somewhat bothersome |
| 3 | Severe | Hot burning sensation that causes definite discomfort |
| | | and may interrupt daily activities or sleep |

Stinging

| | | |
|---|---|---|
| 0 | None | No stinging |
| 1 | Mild | Slight stinging sensation; not really bothersome |
| 2 | Moderate | Definite stinging sensation that is somewhat |
| | | bothersome |
| 3 | Severe | Severe stinging sensation that causes definite |
| | | discomfort and may interrupt daily activities or sleep |

### Example 2

Efficacy:

Co-primary efficacy variables were evaluated in this study, and include the following:
Investigator's Global Assessment (IGA)
Lesion count (separately for inflammatory and non-inflammatory) The co-primary endpoints are:
Proportion of subjects with an assessment of clear or almost clear with at least a 2-grade improvement in IGA at Week 12
Absolute and percent reduction from Baseline in lesion count on the face at Week 12 (separately for inflammatory and non-inflammatory lesions)

Safety:

Safety variables include Investigator Cutaneous Safety Assessment score, subject's tolerability assessment scores, treatment-emergent adverse events (AEs), SAEs, treatment related AEs, AEs leading to study discontinuation, concomitant medications, clinical chemistry, hematology and urinalysis, and ECG evaluation.

Success Criteria:

The following statistical comparisons (both numerically and inferentially) were performed:
Product 3149 (E BPO/E ATRA 3%/0.1%) versus E-BPO 3%, E-ATRA 0.1% and vehicle
Product 3156 (E BPO/E ATRA (3%/0.05%) versus E-BPO 3%, E-ATRA 0.05% and vehicle.

Patient Reported Outcome Questionnaire

The Patient-Reported Evaluation of Facial Acne (PRE-FACE) and Patient Facial Acne Severity Assessment were assessed at study visits 1-6, including screening, baseline, and at Weeks 2, 4, 8, and 12 or early termination (ET), to capture the patient-reported experience of acne vulgaris. The PRE-FACE contains 7-items that constitute two domains. The acne symptom domain (ASD) assesses the severity of acne symptoms (four items) on an 11-point numeric rating scale (NRS) ranging from 0=none to 10=as bad as you can imagine. The acne impact domain (AID) assesses the impacts of acne on the way a patient feels (three items) on an 11-point NRS ranging from 0=not at all to 10=extremely. Higher scores on the Patient Reported Outcome (PRO) questionnaire indicate higher severity of symptoms and impacts associated with acne vulgaris. In addition, the Patient Facial Acne Severity Assessment was assessed along with the PRE-FACE, which is a global item assessing patient-reported overall severity of acne vulgaris on a 5 point verbal rating scale (VRS), ranging from 0 (no acne) to 4 (very severe acne). Respondents were also provided with verbal descriptors to facilitate their ratings.

Results:

TABLE 4
Results for product 3149 at 12 weeks

| | Product 3149 | E-BPO 3% | E-ATRA 0.1% | Vehicle |
|---|---|---|---|---|
| Efficacy | 116 | 118 | 118 | 115 |
| Results | subjects | subjects | subjects | subjects |
| IGA success rate | 39.7% | 22.1% | 31.7% | 12.3% |
| Mean reduction in | 16.9 | 13.8 | 14.9 | 11.5 |
| inflammatory lesions | (64.0%) | (49.4%) | (57.1%) | (42.2%) |
| count | | | | |
| Mean reduction in | 23.6 | 16.2 | 23.8 | 13.7 |
| non-inflammatory | (53.3%) | (37.7%) | (57.1%) | (32.4%) |
| lesions count | | | | |

TABLE 5
Results for product 3149 at 4 weeks

| | Product 3149 | E-BPO 3% | E-ATRA 0.1% | Vehicle |
|---|---|---|---|---|
| Efficacy | 116 | 118 | 118 | 115 |
| Results | subjects | subjects | subjects | subjects |
| IGA success rate | 3.1% | 5.4% | 4.85% | 4.5% |
| Mean reduction in | 8.5 | 8.0 | 8.4 | 7.2 |
| inflammatory lesions | (32.8%) | (29.3%) | (33.2%) | (26.3%) |
| count | | | | |
| Mean reduction in | 11.8 | 7.7 | 10.1 | 7.1 |
| non-inflammatory | (27.6%) | (18.4%) | (25.9%) | (17.9%) |
| lesions count | | | | |

TABLE 6
Results for product 3149 at 8 weeks

| | Product 3149 | E-BPO 3% | E-ATRA 0.1% | Vehicle |
|---|---|---|---|---|
| Efficacy | 116 | 118 | 118 | 115 |
| Results | subjects | subjects | subjects | subjects |
| IGA success rate | 14.9% | 12.7% | 9.3% | 9.4% |
| Mean reduction in | 13.3 | 9.6 | 12.6 | 9.6 |
| inflammatory lesions | (50.4%) | (40.9%) | (49.3%) | (34.1%) |
| count | | | | |
| Mean reduction in | 18.5 | 10.9 | 18 | 12.0 |
| non-inflammatory | (41.9%) | (23.7%) | (43.1%) | (29.2%) |
| lesions count | | | | |

TABLE 7
Results for product 3156 at 12 weeks

| | Product 3156 | E-BPO 3% | E-ATRA 0.05% | Vehicle |
|---|---|---|---|---|
| Efficacy | 117 | 118 | 118 | 115 |
| Results | subjects | subjects | subjects | subjects |
| IGA success rate | 27.4% | 22.1% | 24.9% | 12.3% |
| Mean reduction in | 17.0 | 13.8 | 13.9 | 11.5 |
| inflammatory lesions | (60.8%) | (49.4%) | (51.7%) | (42.2%) |
| count | | | | |
| Mean reduction in | 23.7 | 16.2 | 17.8 | 13.7 |
| non-inflammatory | (54.9%) | (37.7%) | (44.6%) | (32.4%) |
| lesions count | | | | |

TABLE 8
Results for product 3156 at 4 weeks

| | Product 3156 | E-BPO 3% | E-ATRA 0.05% | Vehicle |
|---|---|---|---|---|
| Efficacy | 117 | 118 | 118 | 115 |
| Results | subjects | subjects | subjects | subjects |
| IGA success rate | 3.7% | 5.4% | 1.8% | 4.5% |
| Mean reduction in | 9.3 | 8.0 | 7.2 | 7.2 |
| inflammatory lesions | (31.8%) | (29.3%) | (28.4%) | (26.3%) |
| count | | | | |
| Mean reduction in | 11.4 | 7.7 | 6.8 | 7.1 |
| non-inflammatory | (27.7%) | (18.4%) | (18.8%) | (17.9%) |
| lesions count | | | | |

TABLE 9
Results for product 3156 at 8 weeks

| | | | E-ATRA | |
|---|---|---|---|---|
| | Product 3156 | E-BPO 3% | 0.05% | Vehicle |
| Efficacy Results | 117 subjects | 118 subjects | 118 subjects | 115 subjects |
| IGA success rate | 13.3% | 12.7% | 8.4% | 9.4% |
| Mean reduction | 13.5 (48.5%) | 11.7 (40.9%) | 10.5 | 9.6 |
| in inflammatory | | | (39.6%) | (34.1%) |
| lesions count | | | | |
| Mean reduction | 19.1 (45.4%) | 10.9 (23.7%) | 13.0 | 12.0 |
| in non- | | | (32.4%) | (29.2%) |
| inflammatory | | | | |
| lesions count | | | | |

TABLE 10
Results for product 3149 at 2 weeks

| | | | E-ATRA | Vehicle |
|---|---|---|---|---|
| Patient Reported | Product 3149 | E-BPO 3% | 0.1% | 115 |
| Outcome Results | 116 subjects | 118 subjects | 118 subjects | subjects |
| Mean reduction in | 0.72 | 0.53 | 0.69 | 0.49 |
| Acne Symptom | | | | |
| Domain (ASD) | | | | |
| Mean reduction in | 1.32 | 0.88 | 0.83 | 0.72 |
| Acne Impact | | | | |
| Domain (AID) | | | | |
| Mean reduction in | 0.1 | 0.2 | 0.1 | 0.3 |
| verbal rating scale | | | | |
| (VRS) | | | | |

TABLE 11
Results for product 3149 at 4 weeks

| | | | E-ATRA | Vehicle |
|---|---|---|---|---|
| | Product 3149 | E-BPO 3% | 0.1% | 115 |
| PRO Results | 116 subjects | 118 subjects | 118 subjects | subjects |
| Mean reduction in | 1.21 | 0.96 | 1.18 | 0.78 |
| Acne Symptom | | | | |
| Domain (ASD) | | | | |
| Mean reduction in | 1.97 | 1.51 | 1.44 | 0.92 |
| Acne Impact | | | | |
| Domain (AID) | | | | |
| Mean reduction in | 0.4 | 0.5 | 0.4 | 0.4 |
| verbal rating scale | | | | |
| (VRS) | | | | |

TABLE 12
Results for product 3149 at 8 weeks

| | | | E-ATRA | Vehicle |
|---|---|---|---|---|
| | Product 3149 | E-BPO 3% | 0.1% | 115 |
| PRO Results | 116 subjects | 118 subjects | 118 subjects | subjects |
| Mean reduction in | 1.91 | 1.35 | 1.92 | 1.24 |
| Acne Symptom | | | | |
| Domain (ASD) | | | | |
| Mean reduction in | 2.65 | 2.09 | 2.36 | 1.44 |
| Acne Impact | | | | |
| Domain (AID) | | | | |
| Mean reduction in | 0.6 | 0.5 | 0.5 | 0.6 |
| verbal rating scale | | | | |
| (VRS) | | | | |

TABLE 13
Results for product 3149 at 12 weeks

| | | | E-ATRA | Vehicle |
|---|---|---|---|---|
| | Product 3149 | E-BPO 3% | 0.1% | 115 |
| PRO Results | 116 subjects | 118 subjects | 118 subjects | subjects |
| Mean reduction in | 2.72 | 1.97 | 2.57 | 1.44 |
| Acne Symptom | | | | |
| Domain (ASD) | | | | |
| Mean reduction in | 3.52 | 2.53 | 3.04 | 1.8 |
| Acne Impact | | | | |
| Domain (AID) | | | | |
| Mean reduction in | 1.0 | 0.8 | 0.9 | 0.6 |
| verbal rating scale | | | | |
| (VRS) | | | | |

TABLE 14
Results for product 3156 at 2 weeks

| | | | E-ATRA | Vehicle |
|---|---|---|---|---|
| | Product 3156 | E-BPO 3% | 0.05% | 115 |
| PRO Results | 117 subjects | 118 subjects | 118 subjects | subjects |
| Acne Symptom | 0.72 | 0.53 | 0.79 | 0.49 |
| Domain (ASD) | | | | |
| Acne Impact | 1.71 | 0.88 | 1.03 | 0.72 |
| Domain (AID) | | | | |
| Mean reduction in | 0.4 | 0.2 | 0.2 | 0.3 |
| verbal rating scale | | | | |
| (VRS) | | | | |

TABLE 15
Results for product 3156 at 4 weeks

| | Product | | E-ATRA | Vehicle |
|---|---|---|---|---|
| | 3156 | E-BPO 3% | 0.05% | 115 |
| PRO Results | 117 subjects | 118 subjects | 118 subjects | subjects |
| Acne Symptom | 1.26 | 0.96 | 1.29 | 0.78 |
| Domain (ASD) | | | | |
| Acne Impact | 2.22 | 1.51 | 1.61 | 0.92 |
| Domain (AID) | | | | |
| Mean reduction in | 0.6 | 0.5 | 0.4 | 0.4 |
| verbal rating scale | | | | |
| (VRS) | | | | |

TABLE 16
Results for product 3156 at 8 weeks

| | Product | | E-ATRA | Vehicle |
|---|---|---|---|---|
| | 3156 | E-BPO 3% | 0.05% | 115 |
| PRO Results | 117 subjects | 118 subjects | 118 subjects | subjects |
| Acne Symptom | 2.03 | 1.35 | 1.69 | 1.24 |
| Domain (ASD) | | | | |
| Acne Impact | 3.16 | 2.09 | 2.16 | 1.44 |
| Domain (AID) | | | | |
| Mean reduction in | 0.8 | 0.5 | 0.6 | 0.6 |
| verbal rating scale | | | | |
| (VRS) | | | | |

TABLE 17
Results for product 3156 at 12 weeks

| | Product | | E-ATRA | Vehicle |
|---|---|---|---|---|
| | 3156 | E-BPO 3% | 0.05% | 115 |
| PRO Results | 117 subjects | 118 subjects | 118 subjects | subjects |
| Acne Symptom | 2.68 | 1.97 | 2.40 | 1.44 |
| Domain (ASD) | | | | |
| Acne Impact | 3.94 | 2.53 | 2.82 | 1.8 |
| Domain (AID) | | | | |
| Mean reduction in | 1.1 | 0.8 | 0.8 | 0.6 |
| verbal rating scale | | | | |
| (VRS) | | | | |

TABLE 18
Results for product 3149 at week 12

| Toler- | Product | | | | E-ATRA | | | |
|---|---|---|---|---|---|---|---|---|
| ability | 3149 | | E-BPO 3% | | 0.1% | | Vehicle | |
| Results | 116 subjects | | 118 subjects | | 117 subjects | | 116 subjects | |
| Erythema | 35 | (31.0%) | 24 | (20.3%) | 23 | (19.8%) | 26 | (22.4%) |
| Mild | 18 | (15.9%) | 16 | (13.6%) | 13 | (11.2%) | 19 | (16.4%) |
| Moderate | 15 | (13.3%) | 8 | (6.8%) | 9 | (7.8%) | 7 | (6.0%) |
| Severe | 2 | (1.8%) | 0 | (0.0%) | 1 | (0.9%) | 0 | (0.0%) |
| Scaling | 47 | (41.6%) | 21 | (17.8%) | 54 | (46.6%) | 25 | (21.6%) |
| Mild | 37 | (32.7%) | 14 | (11.9%) | 37 | (31.9%) | 23 | (19.8%) |
| Moderate | 10 | (8.8%) | 7 | (5.9%) | 17 | (14.7%) | 2 | (1.7%) |
| Severe | 0 | (0.0%) | 0 | (0.0%) | 0 | (0.0%) | 0 | (0.0%) |
| Burning | 43 | (38.1%) | 17 | (14.4%) | 41 | (35.3%) | 13 | (11.2%) |
| Mild | 28 | (24.8%) | 15 | (12.7%) | 28 | (24.1%) | 11 | (9.5%) |
| Moderate | 10 | (8.8%) | 2 | (1.7%) | 11 | (9.5%) | 2 | (1.7%) |
| Severe | 5 | (4.4%) | 0 | (0.0%) | 2 | (1.7%) | 0 | (0.0%) |
| Stinging | 35 | (31.0%) | 20 | (16.9%) | 35 | (30.2%) | 16 | (13.8%) |
| Mild | 25 | (22.1%) | 13 | (11.0%) | 23 | (19.8%) | 15 | (12.9%) |
| Moderate | 5 | (4.4%) | 7 | (5.9%) | 10 | (8.6%) | 1 | (0.9%) |
| Severe | 5 | (4.4%) | 0 | (0.0%) | 2 | (1.7%) | 0 | (0.0%) |
| Pig- | 15 | (13.3%) | 12 | (10.2%) | 14 | (12.2%) | 17 | (14.7%) |
| mentation | | | | | | | | |
| Mild | 11 | (9.7%) | 9 | (7.6%) | 10 | (8.7%) | 10 | (8.6%) |
| Moderate | 4 | (3.5%) | 3 | (2.5%) | 3 | (2.6%) | 7 | (6.0%) |
| Severe | 0 | (0.0%) | 0 | (0.0%) | 1 | (0.9%) | 0 | (0.0%) |
| Itching | 28 | (24.8%) | 22 | (18.6%) | 39 | (33.6%) | 22 | (19.0%) |
| Mild | 20 | (17.7%) | 19 | (16.1%) | 29 | (25.0%) | 19 | (16.4%) |
| Moderate | 7 | (6.2%) | 3 | (2.5%) | 10 | (8.6%) | 3 | (2.6%) |
| Severe | 1 | (0.9%) | 0 | (0.0%) | 0 | (0.0%) | 0 | (0.0%) |

TABLE 19
Results for product 3149 at 12 weeks

| Erythema Results | | | | |
|---|---|---|---|---|
| (change in number | | | | |
| of adverse events | Product | | E-ATRA | Vehicle |
| compared to | 3149 | E-BPO 3% | 0.1% | 116 |
| baseline) | 116 subjects | 118 subjects | 117 subjects | subjects |
| week 2, N | 111 | 114 | 115 | 114 |
| Mild | −6 | 5 | −2 | 3 |
| Moderate | 4 | −4 | −1 | −5 |
| Severe | 1 | 0 | 0 | 0 |
| week 4, N | 109 | 114 | 106 | 113 |
| Mild | −7 | 0 | −3 | 6 |
| Moderate | −6 | −7 | −11 | −14 |
| Severe | 1 | 0 | 0 | 0 |
| Week 8, N | 102 | 104 | 98 | 110 |
| Mild | −2 | −3 | −11 | 8 |
| Moderate | −5 | −11 | −13 | −13 |
| Severe | 1 | 0 | 0 | 0 |
| Week 12, N | 97 | 101 | 93 | 102 |
| Mild | −15 | −2 | −13 | 0 |
| Moderate | −14 | −14 | −19 | −17 |
| Severe | 0 | 0 | 1 | 0 |

TABLE 20
Results for product 3149 at 2 weeks

| Toler- | Product | | | | E-ATRA | | | |
|---|---|---|---|---|---|---|---|---|
| ability | 3149 | | E-BPO 3% | | 0.1% | | Vehicle | |
| Results | 116 subjects | | 118 subjects | | 117 subjects | | 116 subjects | |
| Erythema | 35 | (31.0%) | 24 | (20.3%) | 23 | (19.8%) | 26 | (22.4%) |
| Mild | 18 | (15.9%) | 16 | (13.6%) | 13 | (11.2%) | 19 | (16.4%) |
| Moderate | 15 | (13.3%) | 8 | (6.8%) | 9 | (7.8%) | 7 | (6.0%) |
| Severe | 2 | (1.8%) | 0 | (0.0%) | 1 | (0.9%) | 0 | (0.0%) |
| Scaling | 47 | (41.6%) | 21 | (17.8%) | 54 | (46.6%) | 25 | (21.6%) |
| Mild | 37 | (32.7%) | 14 | (11.9%) | 37 | (31.9%) | 23 | (19.8%) |
| Moderate | 10 | (8.8%) | 7 | (5.9%) | 17 | (14.7%) | 2 | (1.7%) |
| Severe | 0 | (0.0%) | 0 | (0.0%) | 0 | (0.0%) | 0 | (0.0%) |
| Burning | 43 | (38.1%) | 17 | (14.4%) | 41 | (35.3%) | 13 | (11.2%) |
| Mild | 28 | (24.8%) | 15 | (12.7%) | 28 | (24.1%) | 11 | (9.5%) |
| Moderate | 10 | (8.8%) | 2 | (1.7%) | 11 | (9.5%) | 2 | (1.7%) |
| Severe | 5 | (4.4%) | 0 | (0.0%) | 2 | (1.7%) | 0 | (0.0%) |
| Stinging | 35 | (31.0%) | 20 | (16.9%) | 35 | (30.2%) | 16 | (13.8%) |
| Mild | 25 | (22.1%) | 13 | (11.0%) | 23 | (19.8%) | 15 | (12.9%) |
| Moderate | 5 | (4.4%) | 7 | (5.9%) | 10 | (8.6%) | 1 | (0.9%) |
| Severe | 5 | (4.4%) | 0 | (0.0%) | 2 | (1.7%) | 0 | (0.0%) |
| Pig- | 15 | (13.3%) | 12 | (10.2%) | 14 | (12.2%) | 17 | (14.7%) |
| mentation | | | | | | | | |
| Mild | 11 | (9.7%) | 9 | (7.6%) | 10 | (8.7%) | 10 | (8.6%) |
| Moderate | 4 | (3.5%) | 3 | (2.5%) | 3 | (2.6%) | 7 | (6.0%) |
| Severe | 0 | (0.0%) | 0 | (0.0%) | 1 | (0.9%) | 0 | (0.0%) |
| Itching | 28 | (24.8%) | 22 | (18.6%) | 39 | (33.6%) | 22 | (19.0%) |
| Mild | 20 | (17.7%) | 19 | (16.1%) | 29 | (25.0%) | 19 | (16.4%) |
| Moderate | 7 | (6.2%) | 3 | (2.5%) | 10 | (8.6%) | 3 | (2.6%) |
| Severe | 1 | (0.9%) | 0 | (0.0%) | 0 | (0.0%) | 0 | (0.0%) |

TABLE 21
Results for product 3156 at week 12

| Toler- | Product | | | | E-ATRA | | | |
|---|---|---|---|---|---|---|---|---|
| ability | 3156 | | E-BPO 3% | | 0.05% | | Vehicle | |
| Results | 117 subjects | | 118 subjects | | 120 subjects | | 116 subjects | |
| Erythema | 30 | (25.9%) | 24 | (20.3%) | 29 | (24.2%) | 26 | (22.4%) |
| mild | 19 | (16.4%) | 16 | (13.6%) | 17 | (14.2%) | 19 | (16.4%) |
| moderate | 10 | (8.6%) | 8 | (6.8%) | 11 | (9.2%) | 7 | (6.0%) |
| severe | 1 | (0.9%) | 0 | (0.0%) | 1 | (0.8%) | 0 | (0.0%) |
| Scaling | 47 | (40.5%) | 21 | (17.8%) | 48 | (40.0%) | 25 | (21.6%) |
| mild | 29 | (25.0%) | 14 | (11.9%) | 30 | (25.0%) | 23 | (19.8%) |
| moderate | 16 | (13.8%) | 7 | (5.9%) | 16 | (13.3%) | 2 | (1.7%) |
| severe | 2 | (1.7%) | 0 | (0.0%) | 2 | (1.7%) | 0 | (0.0%) |
| Burning | 52 | (44.8%) | 17 | (14.4%) | 41 | (34.2%) | 13 | (11.2%) |
| mild | 31 | (26.7%) | 15 | (12.7%) | 23 | (19.2%) | 11 | (9.5%) |
| moderate | 16 | (13.8%) | 2 | (1.7%) | 17 | (14.2%) | 2 | (1.7%) |
| severe | 5 | (4.3%) | 0 | (0.0%) | 1 | (0.8%) | 0 | (0.0%) |
| Stinging | 42 | (36.2%) | 20 | (16.9%) | 31 | (25.8%) | 16 | (13.8%) |
| mild | 30 | (25.9%) | 13 | (11.0%) | 23 | (19.2%) | 15 | (12.9%) |
| moderate | 8 | (6.9%) | 7 | (5.9%) | 8 | (6.7%) | 1 | (0.9%) |
| severe | 4 | (3.4%) | 0 | (0.0%) | 0 | (0.0%) | 0 | (0.0%) |
| Pig- | 16 | (13.8%) | 12 | (10.2%) | 23 | (19.2%) | 17 | (14.7%) |
| mentation | | | | | | | | |
| mild | 13 | (11.2%) | 9 | (7.6%) | 19 | (15.8%) | 10 | (8.6%) |
| moderate | 3 | (2.6%) | 3 | (2.5%) | 4 | (3.3%) | 7 | (6.0%) |
| severe | 0 | (0.0%) | 0 | (0.0%) | 0 | (0.0%) | 0 | (0.0%) |
| Itching | 32 | (27.6%) | 22 | (18.6%) | 35 | (29.2%) | 22 | (19.0%) |
| mild | 25 | (21.6%) | 19 | (16.1%) | 26 | (21.7%) | 19 | (16.4%) |
| moderate | 6 | (5.2%) | 3 | (2.5%) | 8 | (6.7%) | 3 | (2.6%) |
| severe | 1 | (0.9%) | 0 | (0.0%) | 1 | (0.8%) | 0 | (0.0%) |

### CONCLUSIONS

The above results clearly show synergistic effects of the claimed regimens. For example:
Synergistically lower side effects could be observed in week 12 for product 3149 in scaling, stinging, burning, itching (Table 18), for product 3156 in pigmentation, itching (Table 21).
Synergistically higher efficacy could be observed in week 4 for product 3149 in mean reduction in non-inflammatory lesion count (Table 5), week 8 for product 3149 in IGA success rate, mean reduction in non-inflammatory lesion count (Table 6), in week 4 for product 3156 in reduction in inflammatory and non-inflammatory lesion count (Table 8), week 8 for product 3156 in IGA success rate, mean reduction in inflammatory lesion count, mean reduction in non-inflammatory lesion count (Table 9), in week 12 for product 3156 in mean reduction in inflammatory and non-inflammatory lesion count (Table 7).
Surprisingly, both combinations were found to be significantly better than the single active ingredients. Furthermore, the tolerability and safety of the combination therapy and regimen of the invention was shown to have synergistic effect as compared with each composition administered alone.

### Example 3: Efficacy Study of Pharmaceutical Composition Containing Benzoyl Peroxide and Tretinoin for Treatment of Acne Vulgaris

Description: A multi-center, double blind, randomized, vehicle controlled, study of encapsulated 3% benzoyl peroxide and encapsulated 0.1% tretinoin cream (E-BPO/E-ATRA 3%/0.1%) and vehicle cream was performed to assess the efficacy and safety of E-BPO/E-ATRA 3%/0.1% compared to vehicle. Study duration was 12 weeks and included approximately 420 male and female patients afflicted with moderate or severe facial acne vulgaris. Patients were at least 9 years of age and met the inclusion/exclusion criteria described herein.

Dosing: Patients were randomized in a 2:1 ratio to the study product or vehicle treatment group, respectively. Patients applied the study product once daily for 12 weeks on the face (chin, left cheek, right cheek, nose, left forehead and right forehead) in a thin layer, avoiding the eyes, lips, inside the nose, mouth and all mucous membranes.

Clinical and Safety Evaluations were performed at:

1. Visit 1/Screening

2. Visit 2/Baseline, Day 1

3. Visit 3/Week 2, Day 15 (±3 Days)

4. Visit 4/Week 4, Day 29 (±3 Days)

5. Visit 5/Week 8, Day 57 (±3 Days)

6. Visit 6/Week 12, Day 85 (±4 Days)/End of Study or Early Termination.

Patients were admitted into the study after meeting all inclusion/exclusion criteria, including a clinical diagnosis of acne vulgaris and after written informed consent was obtained. Patients with severe acne vulgaris who were appropriate for systemic treatment were counseled regarding their treatment options by the Principal Investigator. Clinician reported efficacy assessments include, at each visit, a 5-point scale Investigator Global Assessment (IGA) ranging from 0 (Clear) to 4 (Severe) of acne assessment, and facial inflammatory and non-inflammatory lesion counts.

Safety was assessed at all visits and included monitoring of adverse experiences; the Investigator Cutaneous Safety Assessment rating of pigmentation, erythema, dryness and scaling on a scale ranging from 0 (None) to 3 (Severe); and the Patient assessment of Local Tolerability rating itching burning and stinging on a scale ranging from 0 (None) to 3 (Severe). A Patient Global Impression of Treatment Side Effects (PGI-SE) was also administered as part of the study design. Standardized optional photography of the face at Baseline and all study visits was performed at select sites.

Urine pregnancy tests were performed for all females of child-bearing potential and premenarchal females, at every visit: Screening, Baseline, Weeks 2, 4, 8 and 12/End of Study or Early Termination. Regardless of the duration of the study, patients that exhibit serious adverse event (SAE) were followed up until the SAE stabilized or resolved, based on investigator's medical judgment.

Evaluation of Efficacy

The investigator performed the Investigator Global Assessment (IGA) and lesion counts (inflammatory and non-inflammatory) at Screening, Baseline, and Weeks 2, 4, 8 and 12/End of Study or Early Termination. Investigators were provided with instructions for IGA assessments and lesion counts to ensure consistency of procedures. Patients having at least 20 and no more than 100 inflammatory lesions (papules, pustules) and at least 30 and no more than 150 non-inflammatory (open and closed comedones) lesions on the face, including the nose, at Screening/Baseline were included in the study. Patients had two (2) or fewer cysts or nodules.

The IGA scale is shown in Table 1 (at Example 1).

Safety Assessments

Safety was evaluated by monitoring incidence of Cutaneous Reactions Assessments, Local Tolerability Assessments, and AEs reporting; at Baseline, at all treatment visits and end-of-treatment visit.

Cutaneous Reactions Assessments

Local application site cutaneous reactions (erythema, dryness, pigmentation and scaling) were assessed by direct evaluation at each study visit except Screening visit. Each of erythema, scaling, dryness and pigmentation was evaluated based on a 0-3 scale (none, mild, moderate, severe) set forth in Table 22 below.

Application site reactions (erythema, scaling, dryness and pigmentation) were not recorded as AEs unless they resulted in the temporary discontinuation of the study product, the discontinuation of the Patient from the study, or the use of a new concomitant medication in order to treat this event.

TABLE 22
Assessments of erythema, sealing, dryness and pigmentation

| | Score | Rating | Definition |
|---|---|---|---|
| Erythema | 0 | None | No erythema |
| | 1 | Mild | Slight pinkness present |
| | 2 | Moderate | Definite redness, easily recognized |
| | 3 | Severe | Intense redness |
| Scaling | 0 | None | No scaling |
| | 1 | Mild | Barely perceptible shedding, |
| | | | noticeable only on |
| | 2 | Moderate | Obvious but not profuse shedding |
| | 3 | Severe | Heavy scale production |
| Pigmentation | 0 | None | No disturbance of oiementation |
| | 1 | Mild | Barely perceptible pigment change |
| | 2 | Moderate | Markedly darker or lighter |
| | 3 | Severe | Complete de-pigmentation or severe |
| | | | hyperpigmention |
| Dryness | 0 | None | No dryness |
| | 1 | Mild | Slight but define roughness |
| | 2 | Moderate | Moderate roughness |
| | 3 | Severe | Marked roughness |

Local Tolerability Assessments

Local application site tolerability was assessed by rating itching, burning and stinging at each study visit except Screening visit. The evaluator determined the score for each variable by asking the patient to grade their experience with each of itching, burning, and stinging over the past 24 hours. Scores were evaluated based on a 0-3 scale (none, mild, moderate, severe) set forth in Table 3 above (at Example 1).

Application site reactions (itching, burning and stinging) were not recorded as AEs unless they result in the temporary discontinuation of the study product, the discontinuation of the Patient from the study, or the use of a new concomitant medication in order to treat this event.

Adverse Events

Intensity of adverse events (“AEs”) was assessed. Mild AEs are usually transient, requiring no special treatment, and do not interfere with Patient's daily activities. Moderate AEs typically introduce a low level of inconvenience or concern to the Patient and can interfere with daily activities but are usually ameliorated by simple therapeutic measures. Severe AEs can interrupt a Patient's usual daily activity and traditionally require systemic drug therapy or other treatment.

The following criteria was used in assessing the apparent causal relationship of an AE to study product:

Definitely—The AE:
follows a reasonable temporal sequence from study product administration
abates upon discontinuation of the study product (de-challenge)
is confirmed by reappearance of the reaction on repeat exposure.

Probably—The AE:
follows a reasonable temporal sequence from study product administration
abates upon discontinuation of the study product (de-challenge).
cannot be reasonably explained by the known characteristics of the Patient's state.

Possible—The AE:
follows a reasonable temporal sequence from study product administration
but that could readily, be produced by a number of other factors.

Unlikely—The AE:
Does not follow a reasonable temporal sequence from the time of study drug administration; and
Was likely produced by other factors such as the subject's clinical state, therapeutic intervention, or concomitant therapy but for which relationship cannot be definitely ruled out.

Not related—The AE:
does not have a reasonable temporal association with the administration of study product
has some other obvious explanation for the event.

Results:

Baseline Characteristics:

The Baseline characteristics were similar among the treatment groups. Patients selected for the treatment groups of this study suffered from moderate and severe acne, with a numerically higher percentage of subjects suffering from moderate acne. The baseline numerical percentage of treatment groups suffering from moderate and severe acne were similar for 5% E-BPO Cream and for Vehicle Cream.

Measurement of Inflammatory Lesion Counts

The results of inflammatory lesion counts of patients groups treated with E-BPO/E-ATRA 3%/0.1% cream and vehicle cream at Baseline, and 2, 4, 8 and 12 weeks are shown in Table 23 below. Table 23 also includes the change in inflammatory lesion counts from baseline for the measurements at 2, 4, 8 and 12 weeks. As shown by the results in Table 23, application of E-BPO/E-ATRA 3%/0.1% demonstrates an early onset of action in treatment of acne. For example, the change from baseline at 2 weeks (27.13% decrease) is significantly better after treatment with E-BPO/E-ATRA 3%/0.1% compared to the change from baseline after treatment with vehicle alone (19.52% decrease).

TABLE 23
Summery of Inflammatory Lesion Counts^{a}

| | | E-BPO/ | |
|---|---|---|---|
| | | E-ATRA | |
| | | 3%/0.1% | Vehicle |
| | | (N = 281) | (N = 143) |
| Baseline | Inflammatory lesion count | 33.5 | 33.5 |
| Week 2 | Inflammatory lesion count | 24.8 | 27.6 |
| | Change from baseline | −8.8 | −5.9 |
| | Percent change from | −27.13% | −19.52% |
| | baseline | | |
| Week 4 | Inflammatory lesion count | 20.2 | 23.8 |
| | Change from baseline | −13.3 | −9.8 |
| | Percent change from | −40.89% | −31.36% |
| | baseline | | |
| Week 8 | Inflammatory lesion count | 16 .5 | 21.2 |
| | Change from baseline | −17.0 | −12.4 |
| | Percent change from | −52.12% | −37.88% |
| | baseline | | |
| Week | Inflammatory lesion count | 12.0 | 18.8 |
| 12 | Change from baseline | −21.5 | −14.7 |
| | Percent change from | −65.63% | −43.45% |
| | baseline | | |

^{a}Summary statistics represent average values, obtained from averaging the summary statistics generated from each imputed dataset. Negative values represent decrease from Baseline. Multiple imputation (MCMC) used to impute missing values.

Measurement of Non-Inflammatory Lesion Counts

The results of non-inflammatory lesion counts of patients groups treated with E-BPO/E-ATRA 3%/0.1% cream and vehicle cream at Baseline, and 2, 4, 8 and 12 weeks are shown in Table 24. Table 24 also includes the change in inflammatory lesion counts from baseline for the measurements at 2, 4, 8 and 12 weeks. As shown by the results in Table 24, application of E-BPO/E-ATRA 3%/0.1% demonstrates an early onset of action in treatment of acne. For example, the absolute reduction in non-inflammatory lesion count from baseline after treatment with E-BPO/E-ATRA 3%/0.1% for about 2 weeks and 4 weeks is 12.4 and 18.6, respectively. Thus, the absolute reduction in non-inflammatory lesion count after about 2 weeks (12.4) is twice the absolute reduction in non-inflammatory lesion count during the period starting at about week 2 and ending at about week 4 of treatment (6.2). The percentage decrease in the number of non-inflammatory lesions after treatment with E-BPO/E-ATRA 3%/0.1% for about 2 weeks is about 25.2%, compared to a percentage decrease in the non-inflammatory lesions of about 17.8% after treatment with vehicle control for 2 weeks.

TABLE 24
Summery of Non-Inflammatory Lesion Counts^{b}

| | | E-BPO/ | |
|---|---|---|---|
| | | E-ATRA | |
| | | 3%/0.1% | Vehicle |
| | | (N = 281) | (N = 143) |
| Baseline | Non-inflammatory lesion count | 48.6 | 47.1 |
| Week 2 | Non-inflammatory lesion count | 36.2 | 39.4 |
| | Change from baseline | −12.4 | −7.7 |
| | Percent change from baseline | −25.24% | −17.78% |
| Week 4 | Non-inflammatory lesion count | 30.0 | 34.4 |
| | Change from baseline | −18.6 | −12.7 |
| | Percent change from baseline | −37.83% | −28.19% |
| Week 8 | Non-inflammatory lesion count | 24.1 | 31.1 |
| | Change from baseline | −24.4 | −16.0 |
| | Percent change from baseline | −50.26% | −34.71% |
| Week 12 | Non-inflammatory lesion count | 18.6 | 28.0 |
| | Change from baseline | −30.0 | −19.1 |
| | Percent change from baseline | −61.49% | −40.37% |

^{b}Summary statistics represent average values, obtained from averaging the summary statistics generated fr om each imputed dataset. Negative values represent decrease from Baseline. Multiple imputation (MCMC) used to impute missing values.

Investigator Global Assessment

The results of the investigator global assessment (IGA) scale of patients groups treated with E-BPO/E-ATRA 3%/0.1% cream and vehicle cream are shown in Table 25 and Table 26, respectively. Affected skin was evaluated at Baseline, and after 2, 4, 8 and 12 weeks. The success and failure analysis are shown in Table 27 and Table 28, where success rates are defined as the proportion of Patients with at least a 2 grade improvement in IGA from Baseline (Table 27), and the proportion of Patients with an assessment of clear or almost clear and with at least a 2 grade improvement in IGA from Baseline (Table 28). As shown by the results in Table 25 through Table 28, application of E-BPO/E-ATRA 3%/0.1% demonstrates an early onset of action in treatment of acne. For example, the success rate (defined as at least a 2-grade improvement in Investigator Global Assessment (IGA) and clear or almost clear) after treatment with treatment with E-BPO/E-ATRA 3%/0.1% for about 2 weeks or 4 weeks is at least twice the success rate after treatment with vehicle control for 2 weeks or 4 weeks, respectively (see Table 28) Also, after 2 weeks, in subjects treated with E-BPO/E-ATRA 3%/0.1%, the percentage of subjects with severe acne decreased from 10.7% to 5.0% (Table 25). Thus, after 2 weeks, more than half of subjects with severe acne treated with E-BPO/E-ATRA 3%/0.1% achieved at least a 1-grade improvement in Investigator Global Assessment. In comparison, in subjects treated with vehicle, the percentage of subjects with severe acne decreased from 7.7% to 5.9% after 2 weeks (Table 26),

TABLE 25
Investigator Global Assessment (E-BPO/E-ATRA 3%/0.1%)^{c}

| | Percentage of patients in IGA category (N = 281) | | | | |
|---|---|---|---|---|---|
| | Clear | Almost clear | Mild | Moderate | Severe |
| Baseline | 0% | 0% | 0% | 89.3% | 10.7% |
| Week 2 | 0% | 1.6% | 25.9% | 67.5% | 5.0% |
| Week 4 | 0% | 5.4% | 42.2% | 47.8% | 4.6% |
| Week 8 | 0.5% | 17.7% | 43.5% | 37.3% | 1.0% |
| Week 12 | 4.1% | 35.9% | 38.3% | 20.5% | 1.3% |

TABLE 26
Investigator Global Assessment (Vehicle)^{c}

| | Percentage of patients in IGA category (N = 143) | | | | |
|---|---|---|---|---|---|
| | Clear | Almost clear | Mild | Moderate | Severe |
| Baseline | 0% | 0% | 0% | 92.3% | 7.7% |
| Week 2 | 0% | 0.7% | 19.0% | 74.4% | 5.9% |
| Week 4 | 0% | 2.4% | 36.8% | 56.4% | 4.5% |
| Week 8 | 0% | 5.6% | 37.8% | 50.6% | 6.0% |
| Week 12 | 0.1% | 14.1% | 37.5% | 44.1% | 4.2% |

TABLE 27
IGA Efficacy Analysis (Success = proportion of patients
with at least 2 grade improvement in IGA from Baseline)^{d}

| | | E-BPO/E-ATRA | Vehicle |
|---|---|---|---|
| | | 3%/0.1% (N = 281) | (N = 143) |
| Week 2 | Success | 2.3% | 1.4% |
| | Failure | 97.7% | 98.6% |
| Week 4 | Success | 8.7% | 3.1% |
| | Failure | 91.3% | 96.9% |
| Week 8 | Success | 21.6% | 7.7% |
| | Failure | 78.4% | 92.3% |
| Week 12 | Success | 43.2% | 15.7% |
| | Failure | 56.8% | 84.3% |

TABLE 28
IGA Efficacy Analysis (Success = proportion of patients with
an assessment at least 2 grade improvement in IGA from Baseline)^{d}

| | | E-BPO/E-ATRA | |
|---|---|---|---|
| | | 3%/0.1% (N = 281) | Vehicle (N = 143) |
| Week 2 | Success | 1.6% | 0.7% |
| | Failure | 98.4% | 99.3% |
| Week 4 | Success | 5.4% | 2.4% |
| | Failure | 94.6% | 97.6% |
| Week 8 | Success | 18.2% | 5.6% |
| | Failure | 81.8% | 94.4% |
| Week 12 | Success | 39.9% | 14.3% |
| | Failure | 60.1% | 85.7% |

Age Subgroups ^{c}Percentages represent average values, obtained from averaging the summary statistics generated from each imputed dataset. Multiple imputation (MCMC) used to impute missing values.^{d}Percentages represent average values, obtained from averaging the summary statistics generated from each imputed dataset. Multiple imputation (MCMC) used to impute missing values.

Subset analysis for age subgroups are shown in Tables 29-31. The absolute change from Baseline in inflammatory lesions and non-inflammatory lesions at Week 12, and success and failure analysis at Week 12 (success rates are defined as the proportion of Patients with an assessment of clear or almost clear and with at least a 2 grade improvement in IGA from Baseline) are shown for subjects that are less than the Median Age (18) and greater than or equal to the Median Age (18) (Table 29), for subjects that are 9-11 years of age and 12-17 years of age (Table 30), and for subjects that are 18-30 years of age and >=31 years of age (Table 31). For example, in subjects 9-11 years old, the success rate (defined as at least a 2-grade improvement in investigator Global Assessment (IGA) and clear or almost clear) after treatment with treatment with E-BPO/E-ATRA 3%/0.1% for 12 weeks was 60.0%, compared with 0% success rate after treatment with vehicle control (see Table 30).

TABLE 29
Absolute change in inflammatory and non-inflammatory lesion count
and IGA Efficacy Analysis for Subjects less than 18 years of age and greater than
or equal to 18 years of age^{e}

| | | Age < Median Age | | Age >= Median Age | |
|---|---|---|---|---|---|
| | | (18 years) | | (18 years) | |
| | | E-BPO/ | | E-BPO/E- | |
| | | E-ATRA | | ATRA | |
| | | 3%/0.1% | Vehicle | 3%/0.1% | Vehicle |
| | | (N = 126) | (N = 60) | (N = 155) | (N = 83) |
| Inflammatory lesion count- | | −21.9 | −12.3 | −21.2 | −16.5 |
| Absolute Change from Baseline | | | | | |
| Non-inflammatory lesion count- | | −31.6 | −15.3 | −28.6 | −21.8 |
| Absolute Change from Baseline | | | | | |
| Assessment of clear or | Success | 39.7% | 11.7% | 40.1% | 16.1% |
| almost clear and with at | Failure | 60.3% | 88.3% | 59.9% | 83.9% |
| least a 2 grade | | | | | |
| improvement in IGA from | | | | | |
| Baseline | | | | | |

TABLE 30
Absolute change in inflammatory and non-inflammatory
lesion count and IGA Efficacy Analysis
for Subjects 9-11 years of age and 12-17 years of age^{e}

| | | Age 9-11 years | | Age 12-17 years | |
|---|---|---|---|---|---|
| | | E-BPO/E- | | E-BPOTE- | |
| | | ATRA | | ATRA | Vehicle |
| | | 3%/0.1% | Vehicle | 3%/0.1% | (N = |
| | | (N = S) | {N = 4) | (N = 121) | 56) |
| Inflammatory lesion count- | | −26.0 | −17.5 | −21.8 | −11.9 |
| Absolute Change from Baseline | | | | | |
| Non-inflammatory lesion count- | | −59.2 | −19.4 | −30.5 | −15.0 |
| Absolute Change from Baseline | | | | | |
| Assessment of clear or | Success | 60.0% | 0.0% | 38.8% | 12.5% |
| almost clear and with | Failure | 40.0% | 100.0% | 61.2% | 87.5% |
| at least a 2 grade | | | | | |
| improvement in IGA | | | | | |
| from Baseline | | | | | |

TABLE 31
Absolute change in inflammatory and non-inflammatory
lesion count and IGA Efficacy Analysis for
Subjects 18-30 years of age and >=31 years of age^{e}

| | | Age 18-30 years | | Age >= 31 years | |
|---|---|---|---|---|---|
| | | E-BPO/E- | | E-BPO/E- | |
| | | ATRA | | ATRA | Vehicle |
| | | 3%/0.1% | Vehicle | 3%/0.1% | (N = |
| | | (N = 120) | (N = 64) | (N = 35) | 19) |
| Inflammatory lesion count- | | −21.9 | −17.5 | 18.9 | −13.1 |
| Absolute Change from Baseline | | | | | |
| Non-inflammatory lesion count- | | −29.8 | −23.1 | −24.8 | −17.6 |
| Absolute Change from Baseline | | | | | |
| Assessment of dear or | Success | 40.8% | 17.5% | 37.7% | 11.6% |
| almost dear and with | Failure | | | | |
| at least a 2 grade | | 59.2% | 82.5% | 62.3% | 88.4% |
| improvement in JGA | | | | | |
| from Baseline | | | | | |

Safety Assessments ^{c}Summary statistics represent average values, obtained from averaging the summary statistics generated from each imputed dataset. Negative values represent decrease from Baseline. Multiple imputation (MCMC) used to impute missing values. Age grouping based on 18 years removed because median age is 18 years.

BPO is a strong oxidizer, and would be expected to result in a significant increase in adverse reactions, such as erythema, scaling, pigmentation, dryness, itching, burning and stinging, in patients being treated for acne with a BPO-containing formulation. As demonstrated in U.S. Application Publication Nos, 2010/0016443, 2017/0281571 and 2018/0147165 and U.S. Pat. Nos. 9,687,465 and 10,420,743, encapsulation of BPO can reduce the irritation associated with BPO, thus, resulting in only a minor increase in subcutaneous reactions compared to the use of un-encapsulated BPO. Following the use of BPO in the studies described herein, the number of subjects patients reporting no and/or almost no adverse reactions generally increased as the use of the drug continued. Moreover, in several examples, the percentage of patients using the drug reporting no reaction was comparable to the percentage of patients using the vehicle. That is, the treatment regimen described herein has an adverse events value similar to the adverse events value of a vehicle control, particularly as the use of the drug continued,

The results of the safety assessments of patients treated with E-BPO/E-ATRA 3%/0.1% cream and vehicle cream are shown in Table 32 through Table 38.

TABLE 32
Assessments of Erythema at each evaluation

| | | E-BPO/E-ATRA | |
|---|---|---|---|
| | | 3%/0.1% | Vehicle |
| Baseline | n | 274 | 139 |
| | 0-None | 56.2% | 56.1% |
| | 1-Mild | 25.5% | 26.6% |
| | 2-Moderate | 16.1% | 17.3% |
| | 3-Severe | 2.2% | 0% |
| Week 2 | n | 265 | 139 |
| | 0-None | 47.9% | 61.2% |
| | 1-Mild | 36.2% | 26.6% |
| | 2-Moderate | 13.6% | 11.5% |
| | 3-Severe | 2.3% | 0.7% |
| Week 4 | n | 263 | 138 |
| | 0-None | 52.1% | 66.7% |
| | 1-Mild | 35.4% | 23.2% |
| | 2-Moderate | 11.8% | 9.4% |
| | 3-Severe | 0.8% | 0.7% |
| Week 8 | n | 254 | 134 |
| | 0-None | 60.6% | 67.9% |
| | 1-Mild | 32.3% | 23.9% |
| | 2-Moderate | 6.7% | 7.5% |
| | 3-Severe | 0.4% | 0.7% |
| Week 12 | n | 250 | 132 |
| | 0-None | 62.0% | 65.9% |
| | 1-Mild | 33.2% | 25.8% |
| | 2-Moderate | 4.4% | 8.3% |
| | 3-Severe | 0.4% | 0% |

TABLE 33
Assessments of Sealing at each evaluation

| | | E-BPO/E-ATRA | |
|---|---|---|---|
| | | 3%/0.1% | Vehicle |
| Baseline | n | 274 | 139 |
| | 0-None | 81.8% | 79.9% |
| | 1-Mild | 14.6% | 16.5% |
| | 2-Moderate | 3.6% | 3.6% |
| | 3-Severe | 0% | 0 |
| Week 2 | n | 265 | 139 |
| | 0-None | 58.5% | 86.3% |
| | 1-Mild | 26.4% | 11.5% |
| | 2-Moderate | 14.0% | 1.4% |
| | 3-Severe | 1.1% | 0.7% |
| Week 4 | n | 263 | 138 |
| | 0-None | 64.3% | 81.9% |
| | 1-Mild | 26.6% | 17.4% |
| | 2-Moderate | 8.0% | 0% |
| | 3-Severe | 1.1% | 0.7% |
| Week 8 | n | 254 | 134 |
| | 0-None | 71.7% | 83.6% |
| | 1-Mild | 24.0% | 14.9% |
| | 2-Moderate | 4.3% | 0.7% |
| | 3-Severe | 0% | 0.7% |
| Week 12 | n | 250 | 132 |
| | 0-None | 78.8% | 83.3% |
| | 1-Mild | 19.6% | 15.9% |
| | 2-Moderate | 1.6% | 0.8% |
| | 3-Severe | 0% | 0% |

TABLE 34
Assessments of Pigmentation at each evaluation

| | | E-BPO/E-ATRA | |
|---|---|---|---|
| | | 3%/0.1% | Vehicle |
| Baseline | n | 274 | 139 |
| | 0-None | 58.4% | 55.4% |
| | 1-Mild | 28.5% | 28.1% |
| | 2-Moderate | 11.3% | 15.1% |
| | 3-Severe | 1.8% | 1.4% |
| Week 2 | n | 265 | 139 |
| | 0-None | 61.9% | 65.5% |
| | 1-Mild | 28.7% | 25.2% |
| | 2-Moderate | 8.7% | 9.4% |
| | 3-Severe | 0.8% | 0% |
| Week 4 | n | 263 | 138 |
| | 0-None | 59.3% | 61.6% |
| | 1-Mild | 30.0% | 30.4% |
| | 2-Moderate | 9.5% | 8.0% |
| | 3-Severe | 1.1% | 0% |
| Week 8 | n | 254 | 134 |
| | 0-None | 64.2% | 65.7% |
| | 1-Mild | 26.0% | 27.6% |
| | 2-Moderate | 7.9% | 6.0% |
| | 3-Severe | 2.0% | 0.7% |
| Week 12 | n | 250 | 132 |
| | 0-None | 61.6% | 67.4% |
| | 1-Mild | 32.8% | 27.3% |
| | 2-Moderate | 4.8% | 5.3% |
| | 3-Severe | 0.8% | 0% |

TABLE 35
Assessments of Dryness at each evaluation

| | | E-BPO/E- | |
|---|---|---|---|
| | | ATRA | |
| | | 3%/0.1% | Vehicle |
| Baseline | n | 274 | 139 |
| | 0-None | 74.1% | 78.4% |
| | 1-Mild | 18.6% | 16.5% |
| | 2-Moderate | 5.8% | 4.3% |
| | 3-Severe | 1.5% | 0.7% |
| Week 2 | n | 265 | 139 |
| | 0-None | 53.2% | 78.4% |
| | 1-Mild | 31.7% | 17.3% |
| | 2-Moderate | 11.7% | 3.6% |
| | 3-Severe | 3.4% | 0.7% |
| Week 4 | n | 263 | 138 |
| | 0-None | 58.9% | 74.6% |
| | 1-Mild | 27.4% | 18.8% |
| | 2-Moderate | 11.0% | 6.5% |
| | 3-Severe | 2.7% | 0% |
| Week 8 | n | 254 | 134 |
| | 0-None | 63.4% | 74.6% |
| | 1-Mild | 28.3% | 19.4% |
| | 2-Moderate | 8.3% | 5.2% |
| | 3-Severe | 0 | 0.7% |
| Week 12 | n | 250 | 132 |
| | 0-None | 71.2% | 78.0% |
| | 1-Mild | 22.0% | 18.9% |
| | 2-Moderate | 6.0% | 3.0% |
| | 3-Seveve | 0.8% | 0% |

TABLE 36
Assessments of Itching at each evaluation

| | | E-BPO/E- | |
|---|---|---|---|
| | | ATRA | |
| | | 3%/0.1% | Vehicle |
| Baseline | n | 274 | 139 |
| | 0-None | 86.5% | 79.9% |
| | 1-Mild | 9.5% | 14.4% |
| | 2-Moderate | 4.0% | 5.0% |
| | 3-Severe | 0% | 0.7% |
| Week 2 | n | 265 | 139 |
| | 0-None | 78.1% | 82.0% |
| | 1-Mild | 16.6% | 15.1% |
| | 2-Moderate | 4.9% | 2.9% |
| | 3-Severe | 0.4% | 0% |
| Week 4 | n | 263 | 138 |
| | 0-None | 78.7% | 82.6% |
| | 1-Mild | 16.3% | 15.2% |
| | 2-Moderate | 3.8% | 2.2% |
| | 3-Severe | 1.1% | 0% |
| Week 8 | n | 254 | 134 |
| | 0-None | 85.8% | 85.1% |
| | 1-Mild | 12.2% | 11.2% |
| | 2-Moderate | 2.0% | 3.7% |
| | 3-Severe | 0% | 0% |
| Week 12 | n | 250 | 132 |
| | 0-None | 86.0% | 89.4% |
| | 1-Mild | 12.8% | 7.6% |
| | 2-Moderate | 1.2% | 3.0% |
| | 3-Severe | 0% | 0% |

TABLE 37
Assessments of Burning at each evaluation

| | | E-BPO/E-ATRA | |
|---|---|---|---|
| | | 3%/0.1% | Vehicle |
| Baseline | n | 274 | 139 |
| | 0-None | 95.3% | 96.4% |
| | 1-Mild | 4.0% | 2.2% |
| | 2-Moderate | 0.4% | 1.4% |
| | 3-Severe | 0.4% | 0% |
| Week 2 | n | 265 | 139 |
| | 0-None | 66.0% | 87.1% |
| | 1-Mild | 18.5% | 11.5% |
| | 2-Moderate | 11.3% | 1.4% |
| | 3-Severe | 4.2% | 0% |
| Week 4 | n | 263 | 138 |
| | 0-None | 79.8% | 92.0% |
| | 1-Mild | 12.9% | 6.5% |
| | 2-Moderate | 5.7% | 1.4% |
| | 3-Severe | 1.5% | 0% |
| Week 8 | n | 254 | 134 |
| | 0-None | 84.3% | 92.5% |
| | 1-Mild | 12.2% | 6.0% |
| | 2-Moderate | 3.5% | 1.5% |
| | 3-Severe | 0% | 0% |
| Week 12 | n | 250 | 132 |
| | 0-None | 92.4% | 95.5% |
| | 1-Mild | 6.0% | 3.8% |
| | 2-Moderate | 1.6% | 0.8% |
| | 3-Severe | 0% | 0% |

TABLE 38
Assessments of Stinging at each evaluation

| | | E-BPO/E- | |
|---|---|---|---|
| | | ATRA | |
| | | 3%/0.1% | Vehicle |
| Baseline | n | 274 | 139 |
| | 0-None | 94.9% | 94.2% |
| | 1-Mild | 4.0% | 3.6% |
| | 2-Moderate | 1.1% | 2.2% |
| | 3-Severe | 0% | 0% |
| Week 2 | n | 265 | 139 |
| | 0-None | 76.6% | 89.9% |
| | 1-Mild | 14.7% | 9.4% |
| | 2-Moderate | 6.0% | 0.7% |
| | 3-Severe | 2.6% | 0% |
| | n | 263 | 138 |
| Week 4 | 0-None | 81.0% | 93.5% |
| | 1-Mild | 14.4% | 5.8% |
| | 2-Moderate | 3.4% | 0.7% |
| | 3-Severe | 1.1% | 0% |
| | n | 254 | 134 |
| Week 8 | 0-None | 87.0% | 95.5% |
| | 1-Mild | 11.4% | 3.7% |
| | 2-Moderate | 1.6% | 0.7% |
| | 3-Severe | 0% | 0% |
| | n | 250 | 132 |
| Week 12 | 0-None | 92.4% | 94.7% |
| | 1-Mild | 7.2% | 3.8% |
| | 2-Moderate | 0.4% | 1.5% |
| | 3-Severe | 0% | 0% |

### Early Onset of Action

The results described herein demonstrate the early onset of action in the treatment of acne with E-BPO/E-ATRA 3%/0.1%. Such early onset of action can improve patient compliance to treatment protocols, resulting in an even higher percentage of success.

Although the exemplary embodiments of the present disclosure have been described in detail with reference to the accompanying examples and drawings, the present disclosure is not limited thereto and can be embodied in many different forms without departing from the technical concept of the present disclosure. Therefore, the exemplary embodiments of the present disclosure are provided for illustrative purposes only and are not intended to limit the technical concept of the present disclosure. The protective scope of the present disclosure should be construed based on any appended claims and combinations thereof, and all the technical concepts in the equivalent scope thereof should be construed as falling within the scope of the present disclosure. As various changes could be made in the above methods and compositions without departing from the scope of the invention, it is intended that all matter contained in the above description shall be interpreted as illustrative and not in a limiting sense. Other embodiments within the scope of the claims herein will be apparent to one skilled in the art from consideration of the specification or practice of the exemplary embodiments disclosed herein. It is intended that the specification be considered exemplary only, with the scope and spirit of the described subject matter being indicated by the claims.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A regimen for providing early onset of action in the treatment of acne comprising topically applying onto an affected skin area of a subject in need thereof, once a day for a period of time of at least about 4, 8 or 12 weeks, a topical medicament which comprises the active agents:
tretinoin or a pharmaceutically acceptable salt thereof, in an amount of about 0.01% to about 0.5% weight; and
benzoyl peroxide in an amount of between about 1% to about 10% weight
and a pharmaceutically acceptable carrier or excipient, wherein the absolute reduction in non-inflammatory lesion count after about 2 weeks is at least about twice the absolute reduction in non-inflammatory lesion count during the period starting at about week 2 and ending at about week 4 of treatment.

2. The regimen of claim 1, wherein the regimen provides a reduction in total number of non-inflammatory lesion counts in a group of such subjects is a reduction of at least about 12 lesions after about 2 weeks of treatment.

3. The regimen of claim 1, wherein the lesion are facial lesions.

4. The regimen of claim 1, wherein the regimen provides, a success rate after about 4 weeks that is at least twice the success rate after treatment with vehicle control for about 4 weeks, wherein the success rate is defined as at least a 2-grade improvement in Investigator Global Assessment (IGA) and clear or almost clear.

5. The regimen of claim 4, wherein the success rate is of about 5.4% after treatment with the pharmaceutical composition for about 4 weeks, compared to a success rate of about 2.4% after treatment with vehicle control for about 4 weeks.

6. A regimen for providing early onset of action in the treatment of acne comprising topically applying onto an affected skin area of a subject in need thereof, once a day for a period of time of at least about 2, 4, 8 or 12 weeks, a topical medicament which comprises the active agents:
tretinoin or a pharmaceutically acceptable salt thereof, in an amount of about 0.01% to about 0.5% weight; and
benzoyl peroxide in an amount of between about 1% to about 10% weight
and a pharmaceutically acceptable carrier or excipient, to achieve a percentage decrease in the number of non-inflammatory lesions of about 25.2% (mean percentage change from baseline) after about 2 weeks, compared to a percentage decrease in the non-inflammatory lesions of about 17.8% after treatment with vehicle control for about 2 weeks.

7. The regimen of claim 6, wherein a reduction in total number of non-inflammatory lesion counts in a group of such subjects is a reduction of at least about 12 lesions after about 2 weeks of treatment.

8. The regimen of claim 6, wherein the regimen provides, in a group of such subjects, a percentage decrease in the number of inflammatory lesions of about 27.1% (mean percentage change from baseline) after about 2 weeks, compared to a percentage decrease in the inflammatory lesions of about 19.5% after treatment with vehicle control for about 2 weeks.

9. The regimen of claim 8, wherein a reduction in total number of inflammatory lesion counts in a group of such subjects is a reduction of at least about 8 lesions after about 2 weeks of treatment.

10. The regimen of claim 6, wherein the regimen provides, in a group of such subjects, a success rate of at least about 2.3% after about 2 weeks, at least about 8.7% after about 4 weeks, at least about 21.6% after about 8 weeks, or at least about 43.2% after about 12 weeks, wherein the success rate is defined as at least a 2-grade improvement in Investigator Global Assessment (IGA).

11. The regimen of claim 10, wherein the success rate after about 2 weeks is at least twice the success rate after treatment with vehicle control for about 2 weeks, wherein the success rate is defined as at least a 2-grade improvement in Investigator Global Assessment (IGA) and clear or almost clear.

12. The regimen of claim 1, wherein the benzoyl peroxide and the tretinoin or a pharmaceutically acceptable salt thereof are the sole active ingredients administered to the subject in need of said treatment during the duration of the regimen.

13. The regimen of claim 6, wherein the benzoyl peroxide and the tretinoin or a pharmaceutically acceptable salt thereof are the sole active ingredients administered to the subject in need of said treatment during the duration of the regimen.

14. The regimen of claim 1, wherein the topical medicament is applied to the face.

15. The regimen of claim 6, wherein the topical medicament is applied to the face.

16. The regimen of claim 1, wherein the acne is any of mild acne, moderate acne, or severe acne.

17. The regimen of claim 6, wherein the acne is any of mild acne, moderate acne, or severe acne.

18. The regimen of claim 1, wherein said topical medicament comprises about 3% w/w of benzoyl peroxide.

19. The regimen of claim 6, wherein said topical medicament comprises about 3% w/w of benzoyl peroxide.

20. The regimen of claim 1, wherein said topical medicament comprises about 0.1% w/w of tretinoin or a pharmaceutically acceptable salt thereof.

21. The regimen of claim 6, wherein said topical medicament comprises about 0.1% w/w of tretinoin or a pharmaceutically acceptable salt thereof.

22. The regimen of claim 1, wherein said topical medicament comprises about 0.1% w/w of tretinoin or a pharmaceutically acceptable salt thereof and about 3% w/w of benzoyl peroxide.

23. The regimen of claim 6, wherein said topical medicament comprises about 0.1% w/w of tretinoin or a pharmaceutically acceptable salt thereof and about 3% w/w of benzoyl peroxide.

24. The regimen of claim 1, wherein said topical medicament is a cream or an emulsion.

25. The regimen of claim 6, wherein said topical medicament is a cream or an emulsion

26. The regimen of claim 1, wherein said benzoyl peroxide is in a form selected from solid or suspension.

27. The regimen of claim 1, wherein the tretinoin or a pharmaceutically acceptable salt thereof is in a form selected from solid, solution or suspension.

28. The regimen of claim 1, wherein said at least one active agent of said medicament is encapsulated in a shell.

29. The regimen of claim 1, wherein each of the active agents, benzoyl peroxide and tretinoin, of the pharmaceutical composition is encapsulated in its own, separate shell.

30. The regimen of claim 27, wherein said shell is an inorganic shell.

31. The regimen of claim 1, wherein said regimen has an adverse events value similar to or lower than the adverse events values of a vehicle control.

32. The regimen of claim 1, wherein the score of at least one parameter evaluated by an Investigator Cutaneous Safety Assessment is similar to or lower than the score of the parameters evaluated with the same treatment regimen with a vehicle control.

33. The regimen of claim 30, wherein said at least one parameter evaluated by the Investigator Cutaneous Safety Assessment is selected from erythema, dryness, scaling, pigmentation and any combinations thereof.

34. The regimen of claim 1, wherein the score of at least one parameter evaluated by a Local Tolerability Score is similar to or lower than the score of the parameter evaluated with the same treatment regimen with a vehicle control.

35. The regimen of claim 32, wherein said at least one parameter evaluated by the Local Tolerability score is selected from Itching, Burning, Stinging, and any combinations thereof.
